(19) United States
(12) Patent Application Publication
(10) Pub. No.: US 2013/0280204 A1
(43) Pub. Date: Oct. 24, 2013

(54) Polymer-Attached Inhibitors of Influenza Virus

(71) Applicant: Massachusetts Institute of Technology, (US)

(72) Inventors: Alisha Weight, Mill Creek, WA (US); Chia Min Lee, Cambridge, MA (US); Alexander M. Klibanov, Boston, MA (US); Jianzhu Chen, Lexington, MA (US)

(21) Appl. No.: 13/839,787

(22) Filed: Mar. 15, 2013

Related U.S. Application Data
(63) Continuation-in-part of application No. 12/197,452, filed on Aug. 25, 2008.
(60) Provisional application No. 60/968,213, filed on Aug. 27, 2007.

(51) Int. Cl. A61K 47/48

(52) U.S. Cl. CPC A61K 47/48315
USPC 424/78.17; 525/436

(57) ABSTRACT

Antiviral compositions containing one or more antiviral agents coupled to a polymer and methods of making and using the compositions, are described herein. The one or more antiviral agents are covalently coupled to the polymer, and thereby prevent or decrease development of drug resistance. In some embodiments, the polymer is a biodegradable polymer. In particular embodiments, the polymer is a water-soluble, biodegradable polymer, which has an overall neutral charge (e.g., no charged groups or overall neutral charge). In a more particular embodiment, the neutral polymer is polyglutamine or a polymer having properties similar to polyglutamine, polyaspartate, and other homopolypeptides that can be modified to have no charge or no net charge. The compositions described herein are effective at treating a variety of viral infections, such as influenza, respiratory syncythial virus, rhinovirus, human metaneumovirus, and other respiratory diseases, while inhibiting or preventing the development of resistance.

### CROSS-REFERENCE TO RELATED APPLICATIONS

This application is a continuation-in-part of U.S. Ser. No. 12/197,452, filed Aug. 25, 2008, which claims benefit of and priority to U.S. Ser. No. 60/968,213, filed on Aug. 27, 2007, both of which are incorporated by reference in their entirety.

### GOVERNMENT SUPPORT

The United States government may have certain rights in this technology by virtue of financial support by the U.S. Army through the Institute for Soldier Nanotechnologies at MIT under Contract DAAD-19-02-D-0002 with the Army Research Office and NIH Grant No. AI074443 (6915739) to Jianzhu Chen.

### FIELD OF THE INVENTION

This invention is generally in the field of polymer compositions which exhibit virucidal and/or virustatic activity.

### BACKGROUND OF THE INVENTION

Influenza A virus causes epidemics and pandemics in human populations, inflicting enormous suffering and economic loss. Currently, two distinct strategies, vaccines and small molecule therapeutics, are used to try to control the spread of the virus. Vaccination offers limited protection, however, and is hampered by several logistical challenges, such as accurately predicting future circulating strains, production of sufficient quantities of vaccines for large populations in a short period of time, and administering the vaccine to populations which are at risk.

With respect to small molecule therapeutics, there are currently four antiviral drugs for the treatment and/or prevention of influenza: amantadine, rimantadine, zanamivir, and oseltamivir. Although these drugs may reduce the severity and duration of influenza infections, they have to be administered within 24-48 hours after the development of symptoms in order to be effective. Further, the emergence of stable and transmissible drug-resistant influenza strains can render these drugs ineffective.

To overcome drug resistance, combination therapies, which contain two or more drugs that simultaneously interfere with different vital processes of a microbe, must be used. Amantadine and rimantadine inhibit the M2 ion channel protein, whereas zanamivir and oseltamivir inhibit the neuraminidase enzyme (NA). Unfortunately, because most of the circulating influenza viruses are already resistant to the M2 inhibitors, traditional combination therapies involving these four drugs have little added value for influenza control. There exists a need for antiviral compositions that are effective in treating viral infections while inhibiting or preventing the development of microbial resistance

Antiviral-polymer conjugates have been described in the literature. For example, Honda et al., Bioorg. Med. Chem., 12, 1929-1932 (2002) described sialidase inhibitors conjugated to polyglutamine. The sialidase inhibitory activities of all polymers prepared against influenza A virus sialidase were less potent than that of zanamivir.

Masuda et al., Chem. Pharm. Bull., 51(12), 1386-1398 (2003) describes the same conjugates described in Honda. As shown in Table 2, the sialidase inhibitory activities of all polymers prepared against influenza A virus sialidase were less potent than that of zanamivir itself.

Whitesides (Whitesides et al., J. Med. Chem., 36, 7780783 (1993), J. Med. Chem., 37, 3419-3433 (1994), and J. Am. Chem. Soc., 118(16), 3789-3800 (1996), and J. Med. Chem., 38, 4179-4190 (1995)) describes polyacrylamides-based conjugates containing a sialic acid bound to the polymer chain. Sialic acid is a hemagglutinin inhibitor, not a neuraminidase (sialidase) inhibitor. Acrylamide is not a biodegradable polymer. Also, acrylamide has been shown to cause toxicity both in vitro and in vivo. Whitesides evaluates efficacy using hemagglutination inhibition assay.

There exists a need to develop biodegradable antiviral compositions that are effective in treating viral infection, such as influenza A and B, while inhibiting or preventing the development of viral resistance, and methods of making and using thereof.

Therefore, it is an object of the invention to provide biodegradable antiviral compositions that are effective in treating viral infection, such as influenza A and B, while inhibiting or preventing the development of viral resistance, and methods of making and using thereof.

### SUMMARY OF THE INVENTION

Antiviral compositions containing one or more antiviral agents coupled to a polymer and methods of making and using the compositions, are described herein. The one or more antiviral agents are covalently coupled to the polymer, and thereby prevent or decrease development of drug resistance. Suitable antiviral agents include, but are not limited to, sialic acid, zanamivir, oseltamivir, laninamivir, peramivir, amantadine, rimantadine, and combinations thereof. In one embodiment, the antiviral agent is a neuraminidase inhibitor, such as zanamivir, oseltamivir, laninamivir, and/or peramivir.

The polymer can be a non-degradable or a biodegradable polymer. In some embodiments, the polymer is a biodegradable polymer. In particular embodiments, the polymer is a water-soluble, biodegradable polymer. Suitable polymers include, but are not limited to, poly(isobutylene-alt-maleic anhydride) (PIBMA), poly(aspartic acid), poly(glutamic acid), polyglutamine, polyaspartate, polylysine, poly(acrylic acid), plyarginic acid, chitosan, carboxymethyl cellulose, carboxymethyl dextran, polyethyleneimine, and blends and copolymers thereof.

In a particular embodiment, the polymer is neutral, i.e., has no charged groups under physiological conditions. In a more particular embodiment, the neutral polymer is polyglutamine or a polymer having properties similar to polyglutamine, polyaspartate, and other homopolypeptides that can be modified to have no charge or no net charge.

The polymers typically have a molecular weight of 1,000 to 1,000,000 Daltons, preferably 10,000 to 1,000,000 Daltons. In some embodiments, the polymer is a neutral polymer, such as a polyglutamine, having a molecular weight from about 50-100 kDa (which is equivalent to about 500 glutamine monomer units). In another embodiment, the compositions contain a physical mixture of a polymer containing one antiviral agent (e.g., neuraminidase inhibitor) and a polymer containing a second antiviral agent (e.g., a second different neuraminidase inhibitor).

The concentration of the antiviral agent(s) is from about 5% to about 25% by weight of the polymer. In one embodiment, the concentration of each antiviral agent is independently 5% by weight of the polymer, 8% by weight of the polymer, 10% by weight of the polymer, 15% by weight of the polymer, 18% by weight of the polymer, 20% by weight of the polymer, or 25% by weight of the polymer.

The antiviral agent(s) can be coupled directly to the polymer by reacting a functional group on the antiviral agent(s) with a functional group on the polymer. Alternatively, the antiviral agent(s) can be coupled to the polymer via a linker. Functional groups on the polymer can be activated in order to facilitate couple of the antiviral agent to the polymer. In some embodiments, the polymer contains functional groups with limited reactivity, e.g., carboxylic groups, which are converted to a more reactive functional group, such as an ester (e.g., benzotriazole ester) in the presence of the antiviral compound or derivative (e.g., containing a linker) to form the conjugate. The resulting conjugated can be treated to remove the more reactive functional groups (e.g., quench with aqueous ammonia to convert groups to an amide). In some embodiments, such treatment results in formation of a neutral polymer backbone.

In those embodiments wherein the inhibitor is conjugated to the polymer via a linker, the linker can be from about 1 to about 10 atoms (e.g., carbons, optionally interrupted with one or more heteroatoms), preferably 1-6 atoms, more preferably 4-6 atoms. In one embodiment, the linker has 5 or 6 atoms, such as 5 or 6 carbons. The bond between the linker and the inhibitor can be a variety of functional groups. In one embodiment, the bond is a carbamate group. In other embodiments, the bond is not an ether bond.

The compositions can be formulated for enteral or parenteral administration. Suitable oral dosage forms include, but are not limited to, tablets, capsules, solutions, suspensions, emulsions, syrups, and lozenges. Suitable dosage forms for intranasal include, but are not limited to, solutions, suspensions, powders and emulsions. Suitable dosage forms for parenteral administration include, but are not limited to, solutions, suspensions, and emulsions.

The compositions described herein are effective at treating a variety of viral infections, such as influenza, respiratory syncythial virus, rhinovirus, human metaneumovirus, and other respiratory diseases, while inhibiting or preventing the development of resistance.

### BRIEF DESCRIPTION OF THE DRAWINGS

FIG. 1 shows the reaction scheme for converting sialic acid to the activated derivative of zanamivir.

FIG. 2 shows the reaction scheme for the synthesis of the O-glycoside of sialic acid.

FIG. 3 shows the chemical structures of zanamivir (1) (FIG. 3A), zanamivir functionalized with three different linkers (2-4) (FIG. 3B), derivatives of polyglutamic acid (5-7) (FIG. 3C), and polyglutamine functionalized with compounds 2-4 (5a-5D, FIGS. 3D and 3E).

FIGS. 4A-4C are graphs showing the IC50 values for low-molecular-weight (3-15 kDa; black bars) and high-molecular-weight (50-100 kDa; white bars) conjugates 5a-7a against (A) Wuhan, (B) TKY, and (C) TKY E119D isolates of influenza A virus. IC50 values, reported as nanomolar concentrations of 1, were determined using the plaque reduction assay after pre-incubation of virus and polymer. Thus, IC50 values reflect inhibition of infection. IC50 values of bare backbones 5-7 ranged from 2 to 39 mM, compared to at least 85 μM for drug conjugates. Thus, the polymers themselves had no appreciable antiviral activity. *p<0.05, **p<0.01, and ***p<0.001 were determined by a two-tailed Student's t-test. All reported values are the mean±SD of at least three independent measurements.

FIG. 5A is a schematic showing the experimental design to determine the efficacy of 5 and 5a in mice. FIG. 5B is a graph showing the viral titers (pfu/mL) from plaque assay of lung homogenates from mice infected with the WSN strain. Equimolar doses of 1 and 5a were used; a 40-fold higher dose of backbone 5 was included as a control. The PBS group was infected and given vehicle only. Mice were dosed intranasally, immediately infected intranasally, and dosed again at 6, 24, and 48 h p.i.; their lungs were harvested 72 h p.i. Mock-infected group, not shown here due to scale, exhibited no plaques. FIG. 5C is a graph showing the viral load in lung homogenates of mice infected with PR8 strain. CT values (higher numbers reflect lower relative levels of viral RNA expression) from qRT-PCR of lung homogenates. The mock group was given only vehicle. Experimental design was the same as in FIG. 5B. *p<0.05 and **p<0.01 were determined by two-tailed Student's t-test. All reported values are the mean of at least three independent measurements.

FIG. 6A is schematic showing the Experimental design to determine therapeutic efficacy of 5a in ferrets. FIG. 6B is a graph showing viral titers in nasal washings of ferrets infected with Nanchang strain of influenza virus. Groups were treated with vehicle (PBS; black bars; n=6), or equimolar doses of 1 (grey bars, n=6) or 5a (white bars, n=6). Statistically significant differences between PBS control and treated groups are represented by *p<0.05 and **p<0.01 as determined by two-tailed Student's t-test. Mean values±SEM represent triplicate measurements.

FIGS. 7A-7C are graphs showing relative binding intensity (%) of polyglutamine (PGN) and polyglutamine-zanamivir conjugate (PGN-ZA) to whole influenza A/WSN/33 virions (FIG. 7A), neuraminidase (NA, FIG. 7B) and hemagglutinin (HA, FIG. 7C) as a function of concentration as determined using ELISA. Bare PGN was included as a control of nonspecific binding and polyglutamine-sialic acid (PGN-SA) as a positive control for HA binding. Error bars represent the SEM from two independent experiments.

FIG. 8A is the experimental design to detect the release of newly synthesized viruses from infected cells. FIG. 8B is a graph showing relative vital titer as function of inhibitor. FIG. 8C is a scheme of a time-of-addition experiment to assay inhibition in the early phase of virus infection in a single replication cycle assay. FIG. 8D is a graph showing the fraction of maximum infection as a function of inhibitor and time of infection. FIG. 8E is a graph showing the IC₅₀ for different inhibitors at different times of administration. Error bars in B, D, and E represent SEM from three to five independent experiments. *P<0.05, **P<0.01, ***P<0.001.

FIG. 9A shows WSN viruses in the presence of absence of 1.8 μM PGN-ZA as visualized by TEM. FIG. 9B is a graph of the fraction of total viral particles for PBS control and PGN-ZA as a function of viral particle distribution.

FIG. 10A is an experimental scheme to study the effect of PGN-ZA on viral binding to target cells and the subsequent endocytosis. FIG. 10B is a series graphs showing virus binding and endocytosis. FIG. 10C is a graph showing the fraction of maximum infection for PBS control and PGN-ZA at different temperatures and in the presence of absence of sialidase.

FIG. 11A is a graph showing normalized virus/cell for PBS control and PGN-ZA as a function of time (min). FIG. 11B is a graph showing viral titer (pfu/ml) for PBS control and two concentrations of PGN-ZA as a function of pH.

FIG. 12 A is a scheme of a drug selection experiment showing the concentration of Zanamivir and PGN-ZA used for virus passage number. The concentration of inhibitor was increased in the subsequent passage by at least two-fold whenever viral appeared to adapt to growing in the presence of the inhibitor. FIG. 12B is a graph showing viral titer on day 3 at each passage as determined by hemagglutination assay with chicken erythrocytes. Each sample was cultured at least in triplicate.

FIG. 13A is sequencing data showing that the E119G mutation emerged in the NA gene in ZA-selected virus at passage 8, and this variant took over the population by passage 12. FIG. 13B is sequencing data showing that residues 119 and 292 of the NA gene in PGN-ZA-selected virus at passage 23 were still wild-type. FIG. 13C is sequencing data showing that amino acid changes in the HA and NA genes of viruses under drug pressure selection in key passages. Changes that were also found in drug-free selected passages are not shown here. N.D.: Sequencing data not available for Passage 9-11 of ZA-selected viruses.

FIGS. 14A and 14B are molecular models showing R220 and D241 residues together with the sialic acid binding site on HA1. The two residues are located at the interface of HA trimers. FIG. 14C is a molecular model showing residue 111 on the structure of the tetramer of A/Tokyo/3/1967NA (Protein Data Bank accession 2BAT). Gly 111 is located at the edge of the interface between NA monomer units. In the wild type NA, Gly 111 contacts with residue 141 of the neighboring unit. If it is substituted by Asp, the residue will face an atomic clash with 141 and the other adjacent residues. Also, the 150-loop on NA is flexible and related to the binding pocket of NA. The G111D may affect the position of the 150-loop, which in turn can present a disruption to the binding site on NA.

### I. DEFINITIONS

“Virucidal”, as used herein, means capable of neutralizing or destroying a virus.

“Virustatic, as used herein, means inhibiting the replication of viruses.

“Biocompatible”, as used herein, means the material does not cause injury, or a toxic or immunologic reaction to living tissue.

“Water soluble polymer”, as used herein, means a polymer having at least some appreciable solubility in water or monophasic aqueous-organic mixtures, e.g., over 1 mg/liter at room temperature.

“IC₅₀”, as used herein, means the concentration of polymer-bound drug to reduce the number of plaques by 50% compared to the number of plaques observed in the absence of polymer-bound rug, both determined by a plaque reduction assay under the same conditions. The IC₅₀ measures the prevention of infection.

“Inhibit or decrease drug resistance”, as used herein, refers to lowering incidence of the emergence of resistant virus or inhibiting influenza viruses that are already resistant to antiviral drugs, such as zanamivir.

“Small molecule”, as used herein, refers to an organic, inorganic, or organometallic antiviral agent having a molecular weight less than 2000, 1500, 1000, 750, or 500 atomic mass units. “Small molecule”, as used herein, does not include biomolecules, such as proteins, enzymes, peptides, nucleic acids, polysaccharides, etc.

“Water-soluble” as used herein, typically means it is completely soluble at inhibitory concentrations.

### II. COMPOSITIONS

Antiviral compositions containing one or more antiviral agents covalently coupled to a water-soluble, biodegradable polymer are described herein. In one embodiment, one or more different antiviral agents, particularly one or more neuraminidase inhibitors, are coupled to a water soluble, biodegradable polymer. In another embodiment, the composition contains a blend of a first water-soluble polymer coupled to a first antiviral agent and a second water-soluble polymer coupled to a second antiviral agent.

A. Antiviral Agents

Any antiviral agent can be used provided that the agent retains some of its activity upon coupling to the polymer. Exemplary classes of antiviral drugs include, but are not limited to, neuraminidase inhibitors, M2 inhibitors, proteinase inhibitors, inosine 5′-monophosphate (IMP) dehydrogenase (a cellular enzyme) inhibitors, viral RNA polymerase inhibitors, and siRNAs. Suitable agents include, but are not limited to, sialic acid, zanamivir, oseltamivir, laninamivir, peramivir, amantadine, rimantadine, and combinations thereof. Zanamivir, oseltamivir, laninamivir, and peramivir inhibit the neuraminidase enzyme (NA), while amantadine and rimantadine inhibit the M2 ion channel protein. Other HA, NA, and/or M2 inhibitors known in the art may also be included. Other inhibitors of NA include fluorosialic acids.

Zanamivir is a relatively small molecule (MW 1,000 Da) that binds to the catalytic site of viral NA to inhibit its activity. Polymers coupled to zanamivir through a covalent linker can be prepared in such a way that the zanamivir moiety in the polymer is still able to bind to the catalytic site and inhibit NA activity. Such polymer-bound antiviral agents should be effective in both inhibiting viral infections, such as influenza, and preventing the emergence of drug resistant viruses. Without being bound by any one theory, it is hypothesized that polymer-bound antiviral agents will be more potent inhibitors than monomer antiviral agent due to multivalent binding. The influenza virion contains 30-50 NA and 300-500 HA molecules. Thus, the presence of multiple copies of inhibitors of NA and/or HA or inhibitors of other targets on the surface of the virus, attached to the same polymer backbone can simultaneously bind to multiple NA and hemagglutinin (HA) and/or other targets on the same virion. This significant increase in the avidity between polymer-bound antiviral moiety and NA/HA should make the polymer-antiviral agent complex a more potent competitive inhibitor. Secondly, because of multivalent binding, the polymer-bound antiviral agent should remain a potent inhibitor of NA/HA even if changes in NA/HA significantly weaken the binding of monomeric antiviral agent to the enzyme's active site. For example, zanamivir binds to the active site of NA with an affinity constant of 10⁻¹⁰ to 10⁻⁹ M (0.1-1.0 nM). Even if the binding affinity is reduced by 10⁶- to 10⁴-fold, the conjugate should still be a potent inhibitor provided that more than three zanamivir moieties attached to the same polymer backbone bind to NA on the same virion at the same time. This is supported by the fact that zanamivir still binds to the catalytic site of NA of most zanamivir resistant viruses (IC₅₀ of 15 to 645 nM). Finally, the binding of a large polymer to multiple NA molecules could create steric hindrance or viral aggregates that interfere with viral infection in addition to the viral release from infected cells.

Coupling two or more other inhibitors, which inhibit influenza virus through a different target, to the same polymer backbone and/or combination of monofunctional polymer-attached ligands may more effectively suppress viral resistance. For example, during influenza virus infection, bonding of hemagglutinin (HA) to sialic acid (SA) residues of glycoproteins on the surface of the cell is critical for viral entry into the cell. Since SA is the cellular receptor for influenza virus, the use of SA itself may help to suppress viral resistance because a viral HA that does not bind sialic acid may have reduced ability to infect host cells.

Both zanamivir and sialic acid exert their effects by binding to particular targets (NA and HA, respectively) on the virion. Therefore, binding these agents to the same polymer backbone may result in a composition that does not need to be taken into the cell to exert its inhibitory effect. Polymers containing zanamivir and/or sialic acid covalently bound to the same polymer backbone or a physical mixture of polymer containing zanamivir and polymer containing sialic acid, may prove to be particularly effective in preventing the emergence of drug-resistant viruses. Zanamivir and sialic acid inhibit influenza virus through different targets and therefore should benefit from combination therapy. Moreover, due to multivalent binding, polymeric inhibitors may remain effective against virus which are resistant to monomeric inhibitors.

The concentration of the antiviral agent is from about 5% to about 25% by weight of the polymer. In one embodiment, the concentration of each antiviral agent is independently 5% by weight of the polymer, 8% by weight of the polymer, 10% by weight of the polymer, 15% by weight of the polymer, 18% by weight of the polymer, 20% by weight of the polymer, or 25% by weight of the polymer. In particular embodiments, the antiviral agent is a neuraminidase inhibitor, such as Zanamivir, having a concentration of about 10% or 10% by weight.

B. Polymers

The one or more antimicrobial agents can be coupled to any water-soluble, biocompatible polymer. In some embodiments, the polymer is biodegradable. In one embodiment, the one or more antimicrobial agents are coupled to the same polymer. In another embodiment, the composition contains a physical mixture of a first antimicrobial agent coupled to a first water-soluble, biocompatible polymer, such as a biodegradable polymer, and a second antimicrobial agent coupled to a second water-soluble, biocompatible polymer, such as a biodegradable polymer. The polymers may be the same polymer (i.e., have the same chemical composition and molecular weight) or different polymers (i.e., different chemical compositions and/or molecular weights).

Suitable polymers include, but are not limited to, poly(isobutylene-alt-maleic anhydride) (PIBMA), poly(aspartic acid), poly(glutamic acid), polyglutamine, polyaspartate, other homopolypeptides which are overall neutral, polylysine, poly(acrylic acid), plyarginic acid, chitosan, carboxymethyl cellulose, carboxymethyl dextran, polyethyleneimine, and blends and copolymers thereof. In one embodiment, the polymer is biodegradable. In another embodiment, the polymer is biodegradable and has an overall neutral charge (e.g., has no charged groups at physiological pH or the overall charge of the groups is neutral). In a particular embodiment, the polymer is polyglutamine.

The antiviral agent(s) are coupled to the polymer via a functional group which is shown not to participate in the binding of the agent to the virus. For example, X-ray crystal structures of zanamivir bound to influenza NA show that the 7-hydroxyl group of the sugar has no direct contact with NA and therefore the attachment of the agent to the polymer via the 7-position should not disrupt the binding interaction. The 7-hydroxyl group can also be converted to other reactive functional groups, such as amino groups or sulfhydryl groups. Therefore, polymers containing functional groups which react with hydroxy, amino, or sulfhydryl groups or groups which are capable of being converted to functional groups which react with hydroxy, amino, or sulfhydryl groups can be used to prepare the compositions described herein. Alternatively, the polymer can contain nucleophilic groups, such as hydroxy, amino, or thiol groups, which react with electrophilic groups on the antimicrobial agent.

In some embodiments, the carboxylic acid groups on polyglutamic acid are activated by converting these groups to more reactive groups. For example, the carboxylic acid groups of polyglutamic acid are converted to benzotriazole ester groups. Acid chlorides and esters are typically more reactive than the corresponding carboxylic acid group.

The polymers typically have a molecular weight of 1,000 to 1,000,000 Daltons, preferably 10,000 to 1,000,000 Daltons. In a particular embodiment, the molecular weight of the polymer is 50-100 kD.

### III. METHOD OF MANUFACTURE

The compositions described herein can be prepared by covalently attaching antiviral agents, or derivative thereof, to a water-soluble, biocompatible polymer, preferably a water-soluble polymer. For example, the antiviral agents to be coupled to the polymer are activated using a variety of chemistries known in the art to form reactive derivatives. The reactive derivative of the antimicrobial agent is reacted with the polymer to covalently link the antiviral agents to the polymer. The reactive derivative can contain a nucleophilic or electrophilic group which reacts directly with an electrophilic group or nucleophilic group on the polymer. Alternatively, the reactive derivative contains a linker which is coupled to the polymer backbone.

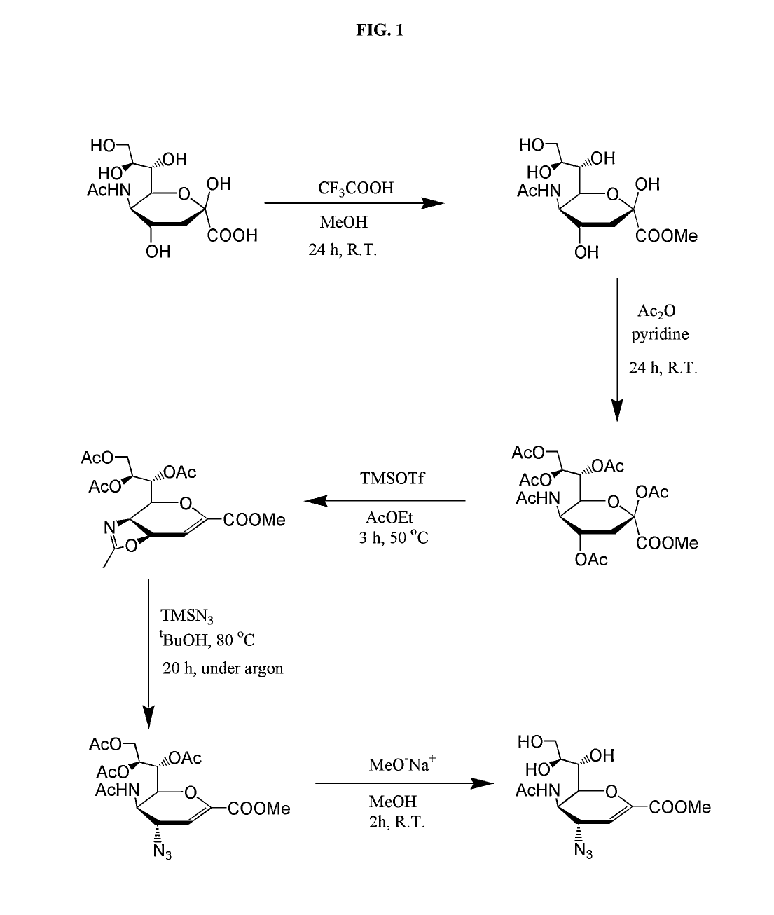

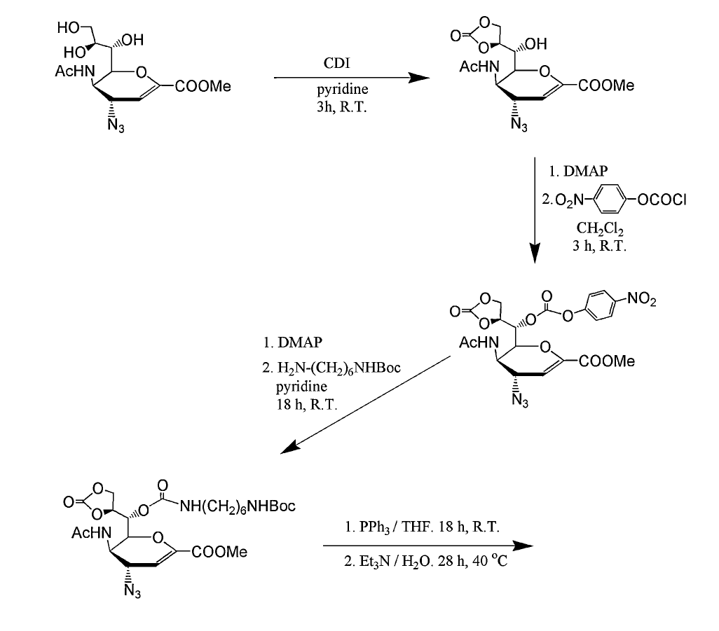

In one embodiment, polyglutamic acid is activated as a benzotriazole ester and reacted the derivative of zanamivir in FIG. 1 to form the conjugate. Quenching of the reaction with aqueous ammonia converts unreacted ester groups to amide groups. The resulting polymer is neutral with no charged side chains. In one embodiment, the antiviral agent is attached to the linker via a carbamate bond. In some embodiments, the bond between the antiviral agent and the linker is not an ether linkage.

The dosage to be administered can be readily determined by one of ordinary skill in the art and is dependent on the age and weight of the patient and the infection to be treated. The amount of antiviral agent molecules to be coupled to the polymer is dependent upon the number of reactive groups on the polymer. For a polyglutamine having a molecular weight of 50,000-100,000 Da (avg. 75,000 Da), 10% derivatization equates to about 30 to about 70 antiviral molecules per polymer chain.

### IV. METHODS OF USE AND ADMINISTRATION

The compositions described herein can be used to treat and/or prevent infections in a mammal, such as a human. Infections to be treated include, but are not limited to, viral infections, such as influenza; bacterial infections; fungal infections; parasitic infections; or combinations thereof. The compositions described herein can be formulated for parenteral or enteral administration. In one embodiment, the infection is a viral infection, such as avian or human influenza A or B. The compositions are effective against wild-type or mutant avian and human influenza viruses. The data in the examples show that the conjugates are effective against four (4) wild-type influenza viruses and three (3) mutant strains of influenza virus.

Unlike conjugates containing sialic acid moieties, the conjugates containing a NA inhibitor do not inhibit red blood cell-virus interactions. These results indicate that PGN-ZA does not inhibit binding of influenza viruses to the target cells or endocytosis of influenza viruses into the target cells. A PGN-ZA induced viral aggregation may lead to a direct virucidal effect or interfere with infection. However, no obvious violation of virus integrity or significant aggregation of viruses caused by PGN-ZA was detected. Nor was any significant effect of PGN-ZA on attachment of viruses to the cell surface and their subsequent endocytosis into target cells observed.

The conjugates described herein more effectively inhibit neuraminidase (sialidase) by at least 10, 25, 50, 75, 100, 150, 200, 250-fold or greater compared to the free neuraminidase inhibitor. For Zanamivir-susceptible strains, the conjugate effectively inhibits neuraminidase (sialidase) by at least 2, 5, 10, 15, 17, 20, or 25-fold or greater compared to the free neuraminidase inhibitor. Zanamivir-resistant strains are inhibited by the same polymer 2000-3000-fold (e.g., 2100-2800-fold) better than by the free inhibitor.

In some embodiments, the conjugates exhibit an IC₅₀ value at least a factor of 5, 10, 100, 100, 1000, 10,000, or 100,000 greater than the free neuraminidase inhibitor against WSN, Wuhan, and/or TKY.

In other embodiments, mice treated with the conjugate exhibited at least a 10, 20, 25, 50, 75, 100, 125, 150, or 200-fold decrease in titers compared to the free neuraminidase inhibitor. For mice infected with PR8, treatment with the conjugated described herein reduced viral load by at least 10, 12, 15, 20, 25, 50, 75, or 100-fold compared to the free neuraminidase inhibitor.

In still other embodiments, the conjugates described herein reduced viral titers in ferrets by at least 10, 15, 20, 25, 30, 50, 75, 100, 125, 150, 200, 250, 300, 350, 400, 450, or 500-fold after 3, 4, 5, or 6 days compared to PBS control.]

The conjugates described herein bind specifically to viral neuraminidase and inhibits both its enzymatic activity and the release of newly synthesized virions from infected cells. In contrast to monomeric ZA, however, the polymer-attached drug inhibits early steps of influenza virus infection, thus contributing to the dramatically increased antiviral potency. This inhibition does not appear to be caused by a direct virucidal effect, aggregation of viruses, or inhibition of viral attachment to target cells and the subsequent endocytosis, but rather appears to be due to interfering with intracellular trafficking of the endocytosed viruses and the subsequent virus-endosome fusion. These findings rationalize the enhanced anti-influenza potency of polymer-conjugated ZA and reveal that attaching the drug to a polymeric chain confers a new mechanism of antiviral action potentially useful for minimizing drug resistance.

Compared to its small-molecule predecessor, PGN-ZA is three to four orders of magnitude more potent in inhibiting influenza virus infection, as determined by plaque reduction assays. It was found that, like ZA, PGN-ZA specifically binds to NA and inhibits its enzymatic activity and the release of the newly synthesized viruses from infected cells. PGN-ZA is more potent in inhibiting virus release than ZA itself, likely due to an increased avidity to NA from polymeric binding and hence an increased inhibition of NA's activity. While inhibition of virus release by PGN-ZA was expected, the observation that PGN-ZA also inhibits an early step of influenza infection is surprising. Compared to the inhibition of virus release, which reduces virus titer by over 90%, inhibition of the early step of influenza infection by PGN-ZA lowers infection by 30-50%, indicating that the former process is still the dominant mechanism of inhibition. More importantly, the effect of the two antiviral mechanisms is more than additive, accounting for the greatly enhanced (1,000 fold) antiviral potency of PGN-ZA over monomeric ZA.

A PGN-ZA-induced viral aggregation may lead to a direct virucidal effect or interfere with infection. However, no obvious deformation of virus integrity or significant aggregation of viruses caused by PGN-ZA was detected. Nor was any significant effect of PGN-ZA on attachment of viruses to cell surface and their subsequent endocytosis into target cells observed. What was observed was the prolonged accumulation of viruses inside the cells, including the perinuclear region. Between the initial endocytosis and virus-endosome fusion to release the viral genomic content into the cytosol, viral particles are transported inside the cell in three separate stages. Stage I lasts for an average of six minutes and is characterized by movement in the cell periphery near the initial site of viral binding. In stage II, the virus-bearing endocytic compartment is transported to the perinuclear region in a few seconds. In Stage III, the virus-bearing endocytic compartment moves around the perinuclear region and undergoes maturation. The maturing endosomes undergo an initial acidification to pH 6, followed by a second acidification to pH 5. Following exposure to the low pH in the endosomes, viral HA undergoes a conformation change leading to fusion of the viral envelope with the endosomal membrane and subsequent release of viral genome into the cytosol.

The finding of accumulation of viral particles inside the cells in the presence of PGN-ZA suggests that PGN-ZA interferes with intracellular trafficking of the endocytosed viruses. Furthermore, the accumulation of viral particles in the perinuclear region from t=15 min onwards suggests a block in virus-endosome fusion. PGN-ZA protects influenza virus from low pH-induced inactivation, i.e., HA does not undergo conformation change in response to lowering pH in the presence of PGN-ZA. Furthermore, most accumulated viral particles did not co-localize with Lysotracker, the marker for acidic cellular compartments, suggesting that a block of acidification of virus-bearing endosomes to pH 5. The combined effect of PGN-ZA on endosome acidification and HA conformation change underscores the inhibition of virus-endosome fusion by PGN-ZA. Intriguingly, some inhibitory effects on viral protein production were still observed when PGN-ZA was added at time 1 h p.i., when most of early infection processes ought to have been completed, raising the possibility that the multivalent PGN-ZA may interfere with additional intracellular processes of infection beyond the initial viral trafficking and virus-endosome fusion.

All existing influenza antivirals have only one mode of action, and a rapid emergence of drug-resistant variants is a major challenge in the control of influenza. The data presented here show that PGN-ZA can synergistically inhibit both viral fusion and release at sub-nM concentrations of ZA. This dual mechanism of inhibition has not been observed among known influenza antivirals and consistent the observation that PGN-ZA remains effective against ZA- or oseltamivir-resistant influenza virus isolates. Multivalent antivirals thus offer an alternative to conventional combination therapy by not only protecting against influenza virus infection but also potentially minimizing the emergence of drug resistance.

A. Dosage Forms

The compositions described herein can be formulated for enteral, parenteral, or topical formulation. In one embodiment, the compositions are formulated for enteral or parenteral administration. The formulations may contain one or more pharmaceutically acceptable excipients, carriers, and/or additives. Methods for preparing enteral and parenteral dosage forms are described in Pharmaceutical Dosage Forms and Drug Delivery Systems, 6^{th} Ed., Ansel et al., Williams and Wilkins (1995).

a. Enteral Dosage Forms

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, pH-modifying agents, preservatives, binders, lubricants, disintegrators, fillers, and coating compositions.

Carrier also includes all components of the coating composition which may include plasticizers, pigments, colorants, stabilizing agents, and glidants. Delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

b. Parenteral Dosage Forms

Suitable parenteral dosage forms include, but are not limited to, solutions, suspension, and emulsions. Formulations for parenteral administration may contain one or more pharmaceutically acceptable excipients including, but not limited to, surfactants, salts, buffers, pH modifying agents, emulsifiers, preservatives, anti-oxidants, osmolality/tonicity modifying agents, and water-soluble polymers.

The emulsion is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal.

Other dosage forms include intranasal dosage forms including, but not limited to, solutions, suspensions, powders, and emulsions. The dosage forms may contain one or more pharmaceutically acceptable excipients and/or carriers. Suitable excipients and carriers are described above.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Materials

Poly-L-glutamic acid sodium salt (MW 50-100 kDa) and all other chemicals, biochemicals, and solvents were purchased from Sigma-Aldrich Chemical Co. (St. Louis, Mo.) unless otherwise noted. Zanamivir was obtained from BioDuro (Beijing, China).

Influenza Viruses

Plaque-purified influenza A/WSN/33 (WSN; H1N1) was cultured in E4HG medium from MDCK cells (ATCC; Manassas, Va.). The cells were passaged in Eagle's minimal essential medium (MEM) containing 10% fetal bovine serum. The A/Turkey/MN/80 virus was propagated in 11-day-old embryonated chicken eggs. The grown viruses were clarified by low-speed centrifugation and concentrated before sucrose gradient purification using a Beckman SW41 rotor at 24,000 rpm. Viruses were resuspended in phosphate-buffered saline (PBS) and stored at −80° C.

Influenza virus strains A/Wuhan/359/95 (Wuhan; H3N2), A/turkey/MN/833/80 (TKY; H4N2), and A/turkey/MN/833/80/E119D drug-resistant mutant (TKY E119D) were obtained from the Centers for Disease Control and Prevention (CDC) (Atlanta, Ga.) and propagated as described in the literature.

Influenza virus A/WSN/33 (WSN), subtype H1N1, was kindly provided by Dr. Peter Palese (Mount Sinai School of Medicine, New York City).

Sucrose-gradient purified influenza A/PR/8/34 (PR8; H1N1) was obtained from Charles River Laboratories in HEPES-saline buffer (Wilmington, Mass.) and diluted with PBS (pH 7.2) before use. Titers were determined by serial titration in the plaque assay.

Stocks of influenza A/Nanchang/933/95 (Nanchang) H3N2 virus were grown in the allantoic cavities of 10-day-old embryonated hens' eggs at 34° C. for 48-72 h. Pooled allantoic fluid was clarified by centrifugation and stored at −70° C. Fifty percent egg infectious dose (EID50) titers were determined by serial titration of virus in eggs and calculated by the method described in the literature.

Assays

Plaque Reduction Assay

The plaque reduction assay to determine inhibitory constants of small-molecule and polymeric inhibitors was performed as previously described.

Virus Binding Assay and HA/NA Specificity Assays

ELISA to measure the direct binding activity of influenza A/WSN/33 virus was performed using a modified literature procedure (36). Briefly, microtiter plates (Corning Polystyrene Universal-BIND Microplate, Corning, N.Y.) were incubated with 50 μL, of varying dilutions of the multivalent inhibitor in PBS at 4° C. overnight and irradiated with 254-nm UV light for 5 min. The solution was then aspirated, and the plates were washed thrice with 2% BSA (Sigma) and 0.05% (v/v) Tween 20 in PBS (PBST), followed by a further 3-h blocking step with 0.3 mL of PBST at RT. The plates were then washed thrice each with PBST and 2% BSA in PBS (PBS-BSA), followed by incubation with a solution containing influenza virus in PBS-BSA at 4° C. overnight. Polyclonal antibodies to the virus diluted in PBS-BSA were subsequently added to the plates and incubated for 5 h at 4° C. The plates were then washed with PBS-BSA thrice and incubated with horseradish peroxidase (HRP)-conjugated secondary antibodies in PBS-BSA for 2 h at 4° C. The plates were washed as above with PBS-BSA before addition of substrate. Colorimetric development of 50 μL of 1-Step Ultra TMB (Thermo Scientific) at RT was stopped with 50 μL of 0.2 M H₂SO₄ after incubation for 30 min, and the absorbance was determined at 450 nm.

For the HA/NA binding specificity assays, the microtiter plates were covalently conjugated with 50 μL of 10 μg/mL polymeric inhibitor and blocked as above. For the HA specificity assay, His-tagged A/WSN/33 (H1N1) HA protein (eEnzyme, Gaithersburg, Md.), primary (mouse anti-His tag IgG, Abcam, Cambridge, Mass.) and secondary (HRP-conjugated goat anti-mouse IgG, Biolegend, San Diego, Calif.) antibodies were mixed in the ratio 4:2:1 and incubated on ice for 20 min. Likewise, His-tagged A/Cal/04/2009 (H1N1) NA (Sin θ Biological, Beijing, China), primary and secondary antibodies were mixed in the ratio 4:2:1, and incubated on ice for 20 min in the NA specificity assay. The mixtures of pre-complexed HA or NA were then diluted to varying concentrations with PBS-BSA, and 50 μL was added to each well and incubated for 2 h at RT. The wells were washed four times with PBST, and HRP activity was measured as in the whole virus binding assay above. The experiment was repeated with varying dilutions of the multivalent inhibitor conjugated to the plate, with concentrations of the pre-complexed proteins constant at 5 μg/mL.

NA inhibition assay was performed as described in the literature.

Virus Release Assay

MDCK cells were incubated with the WSN virus on ice for 60 min to allow binding, and the cells were washed thrice with PBS to remove unbound virus. The cells were then moved to 37° C. to begin the infection process. After 3 h p.i., the infection media was replaced with that containing either PGN-ZA, ZA-linker, or PBS. After 4 h, the supernatant was harvested, and the viral titer quantified by virus plaque assay.

Flow Cytometry

To quantify single-cycle infection by flow cytometry, MDCK cells were infected with WSN virus at moi of 20 for 1 h on ice, followed by washing thrice with ice-cold PBS to remove unbound virus. Infection medium was then added and the temperature raised to 37° C. to allow infection to begin. The inhibitors were added at −1, 0, or 1 h p.i. To remove them, the cells were washed 4 times with pre-warmed PBS. Mock-infected and WSN-infected/untreated (PBS) samples acted as controls. At 3 h p.i., the MDCK cells were trypsinized, washed with PBS twice, and fixed with 2% paraformaldehyde in PBS. The fixed cells were washed with PBS containing 2% FBS (PBS-FBS) twice and resuspended in 0.1% saponin in PBS-FBS (permeabilization buffer). After 10 min at RT, the samples were centrifuged and resuspended in 80 μL of the permeabilization buffer containing 1 μg/mL anti-NP (AbD Serotec, Raleigh, N.C.) and anti-M1 (Abcam) monoclonal antibodies. Following a 1-h incubation in the dark at RT, unbound antibodies were removed by two washes with 1 mL of the permeabilization buffer. The cells were then incubated with 50 μL of phycoerythrin-linked anti-mouse IgG antibody (Biolegend) for 30 min at RT. Unbound antibodies were again removed by two washes of 1 mL of the permeabilization buffer. Finally, the cell pellets were resuspended in PBS-FBS and analyzed on the Accuri C6 flow cytometer. The analytical gatings between infected and uninfected cells were determined from the PE fluorescence intensity histograms of the mock-infected negative controls. The extent of influenza infection was quantified as the fraction of cells with fluorescence intensity above the analytical gating. All samples were normalized to the mean of 3 infected, untreated (PBS) controls.

For the flow cytometry-based binding and internalization studies, MDCK cells were trypsinized, resuspended in DMEM, and exposed to WSN virus at moi of 20 on ice for 1 h. For internalization studies, the cells were then moved to 37° C. for 30 min to allow endocytosis of the bound virions. To differentiate between internalized and surface-bound virions, bacterial sialidase was introduced to remove surface-bound virions. The cells were washed twice with DMEM to remove unbound viruses and inhibitor and treated with Arthrobacter ureafaciens (20 mU/100 μL) and Vibrio cholera (25 mU/100 μL) neuraminidase for 1 h at 37° C. For subsequent flow cytometry analysis, the cells were fixed, processed, and analyzed as described above.

Virus Plaque Assays

Virus plaque assays were performed using a modified literature procedure. Briefly, for the early-stage inhibition samples (−1 to 1 h), equal volumes of viruses and inhibitors of various concentrations were pre-incubated for 1 h prior to cell inoculation. Thereafter the inoculum was aspirated, and the cells were washed 4 times with pre-warmed PBS to remove any residual inhibitor or viruses. The cells were then overlaid with agar solution with no inhibitor. In the case of the late-stage inhibition samples (1 to 72 h), there was no pre-treatment, and the initial 1-h infection was also done in the absence of inhibitors. After infection, the cells were overlaid with agar solution containing the appropriate concentrations of inhibitor. For the combination samples (−1 to 72 h), the inhibitor was present throughout the assay, from pre-treatment through the agar overlay.

Transmission Electron Microscopy (TEM)

The WSN virus was sonicated, and remaining viral aggregates were removed using a 0.2-μm-pore filter. The virus was then incubated at RT for 1 h with either PBS or PGN-ZA at a concentration exceeding 10×IC₅₀ (1.8 μM of ZA). The surface of a carbon/formvar film supported on a Cu grid was treated with a drop of the influenza virus solution for 1 min. The surface was washed by successively dipping the grid in 3 drops of water and stained with either 0.75% uranyl acetate or 1% phosphotungstic acid. A drop of the stain was placed on the surface of the grid for 45 and then removed by absorption onto a piece of filter paper. The samples were allowed to dry overnight and analyzed using a Tecnai G² Spirit Biotwin TEM instrument.

Fluorescence Microscopy

The labeling process was modified from a published protocol. The WSN virus was labeled with Alexa Fluor 647 carboxylic acid succinimidyl ester dye (Invitrogen, Grand Island, N.Y.) in a carbonate buffer (pH 9.3) at RT for 1 h with gentle shaking Unbound dye was removed by a buffer exchange with 50 mM Hepes buffer (pH 7.4, 145 mM NaCl) using Nap5 gel filtration columns (GE Healthcare, Waukesha, Wis.). Viral aggregates were removed by filtration immediately prior to experiments using a 0.2-μm filter. MDCK cells were exposed to the dye-labeled WSN at moi of 20 on ice for 1 h to allow binding. Unbound virus and inhibitors were removed by 3 washes of cold PBS. Medium containing Lysotracker (Invitrogen) and either PBS or PGN-ZA were added to the samples before they were immediately moved to a 37° C. water bath to begin infection. Samples were washed, fixed at 0, 5, 15, 30, and 60 min p.i. with 2% paraformaldehyde, and the cell boundaries labeled with GFP-tagged E-cadherin. The samples were then cured overnight with DAPI Prolong Gold (Invitrogen). Images were taken on Applied Precision DeltaVision Ultimate Focus Microscope with a 60× objective. The images taken were deconvolved to visualize individual virus peaks. For quantitative analysis, the number of viruses per cell was quantified using ImageJ and normalized to the PBS controls. Amantadine, an inhibitor of the M2 ion channel, was used as a positive control. The assay was done the same way except that Alexa Fluor 488-labeled TKY virus and 125 μM amantadine were used. The TKY virus had to be used because WSN is resistant to amantadine.

Inactivation of Virus by Acidic Treatment

The low-pH inactivation of influenza virus was performed as previously described and the virus was titrated with the plaque assay.

Statistical Analysis was performed using two-tailed t-tests (40).

### Example 1

### Synthesis and Characterization of Zanamivir-Polyglutamine Conjugates

Zanamivir derivative (2) was synthesized as described in the literature.

### 3-Acetamido-2-(1-(((2-(2-aminoethoxy)ethyl)carbamoyl)oxy)-2,3-dihydroxypropyl)-4-guanidino-3,4-dihydro-2H-pyran-6-carboxylic acid (3)

(3) was synthesized using a modified literature procedure with tert-butyl-(2-(2-aminoethoxy)ethyl)carbamate (ChemPep, Wellington, Fla.) to introduce the linking group. Subsequent reduction/deprotection with triphenyl phosphine/triethylamine/H₂O and guanidinylation with N,N′-bis-tert-butoxycarbonyl-1H-pyrazole-1-carboxamidine of intermediates were performed as previously described, with modification to the purification schemes. For both intermediates, purification was done using a reverse-phase silica plug (Sep Pak C18 cartridge vac 6 cc, Waters, Milford, Mass.). Crude intermediates were loaded in water and 1:4 H₂O:methanol, respectively. Product was eluted with 12 mL of water followed by either 15% acetonitrile or 40% methanol, respectively. BOC-deprotection was performed to give compound 3.

1H NMR (D₂O) δ (500 MHz)-1.85 (3H, s, CH₃CONH), 3.05-3.25 (4H, m, —NHCH₂CH₂OCH₂CH₂NH₂), 3.40 (1H, dd, H-9a), 3.50 (2H, m, —NHCH₂CH₂OCH₂CH₂NH₂), 3.60 (1H, d, H-9b), 3.65 (2H, m, —NHCH₂CH₂OCH₂CH₂NH₂), 3.95 (1H, m, H-8), 4.05 (1H, t, H-5), 4.35 (1H, d, H-4), 4.45 (1H, d, H-6), 4.90 (1H, d, H-7), 5.95 (1H, d, H-3).

### 3-Acetamido-2-(1-(((4-(aminomethyl)phenyl)carbamoyl)oxy)-2,3-dihydroxypropyl)-4-guanidino-3,4-dihydro-2H-pyran-6-carboxylic acid (4)

(4) was synthesized analogously to 3 above using tert-butyl-4-aminobenzylcarbamate to introduce the linker group.

1H NMR (D₂O) δ (500 MHz)-1.85 (3H, s, CH₃CONH), 3.45 (1H, q, H-9a), 3.60 (1H, d, H-9b), 4.10-4.20 (2H, m, H-5 and H-8), 4.35-4.45 (3H, m, PhCH₂NH₂ and H-4), 4.50 (1H, d, H-6), 5.0 (1H, d, H-7), 5.85 (1H, d, H-3), 7.25-7.35 (4H, m, aromatic).

To prepare polymer conjugate 5a, 2 was reacted with the benzotriazole ester of polyglutamic acid, followed by quenching with NH₄OH. Zanamivir content was quantified by ¹H NMR.

Polymer Conjugates 5b and 5c

5b and 5c were synthesized analogously to 5a.

1H NMR (D₂O) δ (500 MHz)—For 5b: 1.8-2.1 (5H, m, 2H polymer and CH₃CONH), 2.1-2.4 (2H, d, 2H polymer), 3.0-3.2 (4H, m, NHCH₂CH₂OCH₂CH₂NH₂), 3.35-3.45 (2H, m, NHCH₂CH₂OCH₂CH₂NH₂), 3.45 (1H, dd, H-9b), 3.55 (1H, d, H-9a), 3.6 (2H, m, NHCH₂CH₂OCH₂CH₂NH₂), 3.95 (1H, m, H-8), 4.05-4.25 (2H, s, 1H polymer and H-5), 4.45 (1H, d, H-4), 4.55 (1H, d, H-6), 5.7 (1H, s, H-3). For 5c: 1.9-2.1 (5H, m, 2H polymer and CH₃CONH), 2.2-2.4 (2H, d, 2H polymer), 3.5 (1H, q, H-9a), 3.6 (1H, dd, H-9b), 4.1-4.35 (9H, m, 4H polymer, H-4, H-5, H-8, PhCH₂NH₂), 4.5 (1H, d, H-6), 5.7 (1H, d, H-3), 7.3 (4H, m, 4H aromatic).

Conjugate 6a and Scaffold 6

Conjugate 6a was synthesized analogously to 5a using 3 mL of 0.1 M NaOH to quench the polymer conjugation reaction for 48 h at RT. Un-derivatized 6 was synthesized by quenching benzotriazole-activated poly-L-glutamate with an excess of 0.1 M NaOH. After quenching, both reactions were diluted with distilled water and buffer exchanged into the same at least four times using an Amicon Ultra Centrifugal Filter with an Ultracel regenerated cellulose membrane (15 mL, 15 kDa MW cutoff) at 4,000×g before lyophilization.

1H NMR (D₂O) δ (500 MHz)

6a: 1.1-1.4 (8H, m, —NHCH2(CH₂)₄CH₂NH₂), 1.7-2.4 (7H, m, 4H polymer and CH₃CONH), 2.8-3.05 (4H, m, —NHCH₂(CH₂)₄(CH₂NH₂), 3.5 (1H, dd, H-9b), 3.6 (1H, d, H-9a), 3.8 (2H, m, H-5, H-8), 4.1-4.25 (1H, s, 1H polymer), 4.45 (1H, d, ZA), 4.55 (1H, d, H-6), 5.0 (1H, d, H-7), 5.75 (1H, s, H-3). 6: 1.7-2.3 (4H, m, 4H polymer), 4.2 (1H, s, 1H polymer).

Conjugate 7a and Scaffold 7

Attachment of 2 to the activated polymer scaffold was performed analogously to 5a. After 4 h, the reaction was cooled on an ice bath. Solid N-ethyl-N′-(3-dimethylaminopropyl)carbodiimide hydrochloride (2.72 mg, 0.0142 mmol, 1.2 eq) was added to the reaction mixture, which was subsequently purged with argon gas. A solution of 4-dimethylaminopyridine (0.072 mg, 0.00059 mmol, 0.05 eq) in DMF (15 μL) was then added, and the mixture was stirred for 5 min. A pre-cooled solution of choline chloride (6.59 mg, 0.0472 mmol, 4 eq) in formamide (240 μL to give a final concentration of formamide in reaction of 30% v/v) was then added dropwise. The reaction was stirred on ice for 16 h and allowed to warm to RT overnight. Bare 7 was synthesized by directly quenching benzotriazole-activated poly-L-glutamate with choline chloride in formamide as described above. After quenching, both reactions were diluted with distilled water and buffer exchanged into the same at least four times using an Amicon Ultra Centrifugal Filter with a regenerated cellulose membrane (15 mL, 15 kDa MW cutoff) at 4,000×g before lyophilization.

1H NMR (D₂O) δ (500 MHz)

7a: 1.2-1.5 (8H, m, —NHCH₂(CH2)₄CH₂NH₂), 2.0-2.8 (7H, m, 4H polymer and CH₃CONH), 2.9-3.05 (4H, m, —NHCH₂(CH₂)₄CH₂NH₂), 3.2 (9H, s, —N(CH₃)₃), 3.5 (1H, dd, H-9a), 3.55 (1H, d, H-9b), 3.7 (2H, s, —CH₂N—) 4.05 (2H, m, H-8 and H-5), 4.1-4.3 (2H, 1H polymer and H-4), 4.5 (1H, d, H-6), 4.55 (2H, s, —CH₂O—), 5.65 (1H, s, H-3).

7: 2.0-2.8 (4H, m, 4H polymer), 3.25 (9H, s, —N(CH₃)₃), 3.8 (2H, s, —CH₂N—), 4.15-4.4 (1H, s, 1H polymer), 4.65 (2H, s, CH₂O—).

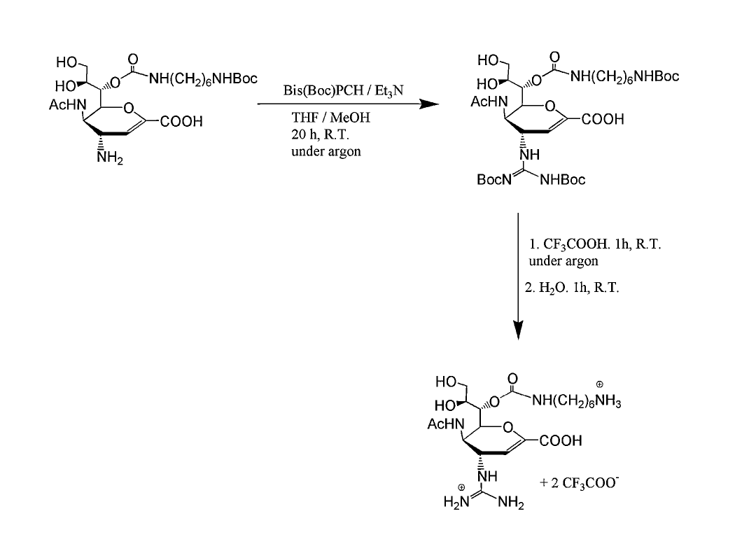

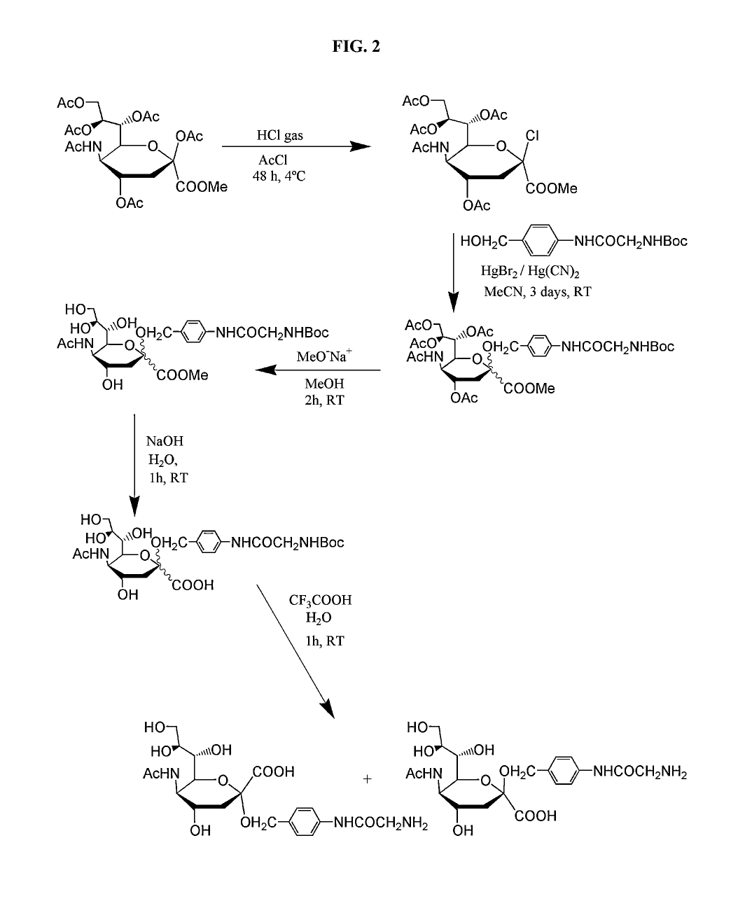

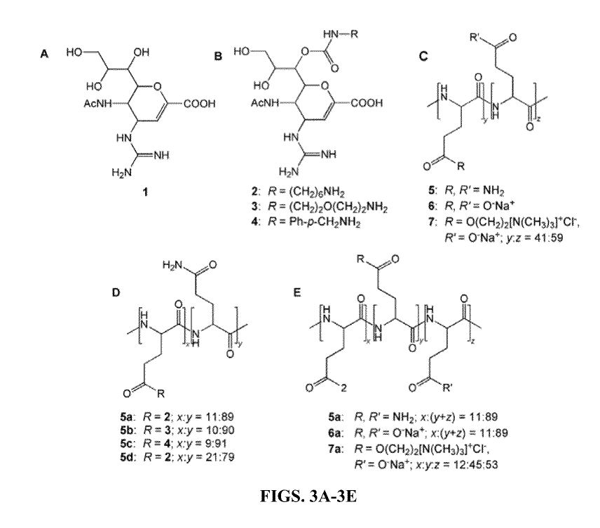

Structures of the compounds referred to below are shown in FIG. 3.

Results and Discussion

The analogs of 1 were evaluated in the plaque reduction assay against three distinct strains of influenza virus: influenza A/WSN/33 (WSN), human influenza A/Wuhan/359/95 (Wuhan), and avian influenza A/turkey/MN/833/80 (TKY). For all three strains, IC50 (half of maximal inhibitory concentration) values showed that 2 was the most potent inhibitor by up to 6-fold over flexible hydrophilic analog 3 and 50-fold over rigid analog 4 (Table 1).

TABLE 1
Antiviral activities of 1's analogs 2-4 and their polymer-attached
derivatives 5a-5c against three strains of influenza A virus,
as determined by the plaque reduction assay.

| In- | IC₅₀ (nM 1)^{a} | | |
|---|---|---|---|
| hibitor | WSN | Wuhan^{b} | TKY^{b} |
| 2 | (1.0 ± 0.4) × 10⁴ | (4.3 ± 0.20) × 10⁵ | (4.3 ± 1.1) × 10⁴ |
| 3 | (5.7 ± 3.9) × 10⁴ | (1.3 ± 0.56) × 10⁶ | (1.4 ± 0.34) × 10⁶ |
| 4 | (7.6 ± 1.3) × 10⁴ | (8.4 ± 1.3) × 10⁵ | (2.3 ± 0.71) × 10⁶ |
| 5a | (1.8 ± 0.5) × 10² | 21 ± 7.3 | (1.8 ± 0.20) × 10² |
| 5b | (1.3 ± 0.80) × 10² | (1.5 ± 0.63) × 10² | (1.5 ± 0.70) × 10³ |
| 5c | 81 ± 1.0 | 43 ± 23 | (9.8 ± 1.2) × 10³ |

^{a}Reported values were determined from experiments run at least in triplicate. The IC₅₀ values (±SD) are expressed as nanomolar concentrations of 1, whether free or conjugated to poly-L-glutamine. To determine the IC₅₀ values, inhibitor and influenza viruses were incubated together prior to the plaque assay. Therefore, the IC₅₀ values reflect inhibition of infection. The IC₅₀ values for unmodified poly-L-glutamine ranged from 2 to 14 mM (on a monomer basis), indicating that the polymer itself had no appreciable antiviral activity.

When attached to the poly-L-glutamine backbone, compound 5a—the polymeric conjugate of 2 (FIG. 3D)—was at least as good as either 5b or 5c against Wuhan, but not necessarily WSN virus, and approximately 10-fold more potent against TKY virus (Table 1). Thus, analog 2 was selected, which is flexible and moderately hydrophobic, for subsequent SAR studies of the polymeric conjugates.

Additional data against other strains as function of percent zanamivir is shown in Table 2.

TABLE 2
IC₅₀ (nM zanamivir)³

| Strain | 1 | 2^{c} | 3 | 4 | 5 |
|---|---|---|---|---|---|
| A/Wuhan/359/95 | (2.3 ± 1.6) × | (4.3 ± 0.2) × | (1.8 ± 0.5) × | 21 ± 7 | (3.4 ± 1.0) × |
| | 10⁴ | 10⁵ | 10₂ | | 10² |
| A/Wuhan/359/95 | (4.8 ± 1.5) × | (3.1 ± 0.1) × | (7.7 ± 5.1) | 51 ± 5 | (1.0 ± 0.6) × |
| | 10⁴ | 10⁵ | 10² | | 10³ |
| A/turkey/MN/833/80 | (2.1 ± 0.9) × | (4.3 ± 1.1) × | (1.2 ± 0.4) × | (1.8 ± 0.2) × | (9.6 ± 5.9) × |
| | 10⁴ | 10⁴ | 10³ | 10² | 10² |
| A/turkey/MN/833/90 | (1.8 ± 0.8) × | >3.6 × 10⁶ | (6.2 ± 4.0) × | (1.7 ± 0.8) × | (2.6 ± 1.5) × |
| E119D | 10⁷ | | 10³ | 10³ | 10³ |
| A/turkey/MN/833/80 | n.d.^{b} | >3.6 × 10⁶ | (6.6 ± 3.3) × | (3.6 ± 1.3) × | (3.0) ± 1.2) × |
| E119G | | | 10³ | 10³ | 10⁴ |

1 is free zanamivir, 2 is zanamivir plus linker, and 3, 4, and 5 are PGN-ZA, having 5%, 10%, and 20% zanamivir, respectively. Conjugate 4 exhibited a 10,000-fold enhancement against Wuhan WT compared to ZA-linker and at least a 1,000-fold enhancement against Wuhan E119V, turkey.MN E119D, and turkey/MN E119G compared to ZA-linker.

The effect of the length and charge of the polymeric backbone on antiviral Activity was also evaluated. Specifically, a scaffold of poly-L-glutamate of either 3-15 kDa (˜20-100 repeating units) or 50-100 kDa (˜330-660 repeating units) was used, and to it conjugated 10 mole-percent of 2.

Subsequently, the glutamate side chains were modified with ammonia or choline groups to impart a neutral or zwitter-ionic charge state, respectively (FIG. 1E). The inhibitory potency of these six polymeric conjugates was assessed in the plaque assay with Wuhan, wild-type TKY, and 1-resistant (E119D) TKY influenza strains.

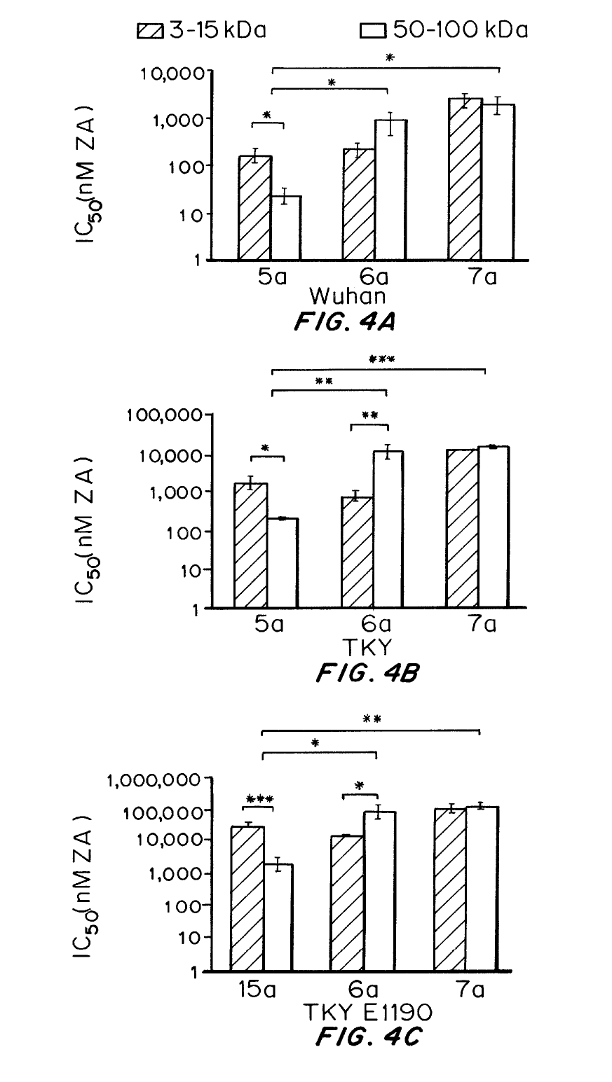

Conjugate 7a, regardless of molecular weight or virus strain, had the highest IC50 value (FIG. 4). For negatively charged conjugate 6a, the low-molecular-weight inhibitor had an IC50 up to 4-fold lower than the high-molecular-weight conjugate against all three viruses. Conversely, high-molecular-weight 5a was up 15-fold more potent than the low-molecular-weight variant and up to 75-fold more potent compared to corresponding charged analogs 6a and 7a.

Activity as a function of linker is shown in Table 3. The alkylene linker was generally the most effective although the benzyl linker showed good activity against Wuhan H3N2 and WSN.

TABLE 3
IC50 (nM Sugar)

| | | PLGN-ZA- | | PLGN- | | PLGN- |
|---|---|---|---|---|---|---|
| Strain | ZA-linker | linker | etherZA | etherZA | aroZA | aroZA |
| Wuhan | (4.3 ± 0.2) × | 21 ± 7 | (1.3 ± 0.56) × | (1.5 ± 0.63) × | (8.4 ± 1.3) × | 43 ± 23 |
| (H3N2) | 10⁵ | | 10⁶ | 10² | 10⁵ | |
| Fold | 2 × 10⁴ | | 9 × 10³ | | 2 × 10⁴ | |
| improvement | | | | | | |
| WSN | (1.0 ± 0.4) × | (1.8 ± 0.5) × | (5.7 ± 3.9) × | (1.3 ± 0.8) × | (7.6 ± 1.3) × | 81 ± 1 |
| (H1N1) | 10⁴ | 10² | 10⁴ | 10² | 10⁴ | |
| Fold | 50 | | 4 × 10² | | 2 × 10² | |
| improvement | | | | | | |
| Turkey/WT | (4.3 ± 1.1) × | (1.8 ± 0.2) × | (1.4 ± 0.34) × | (1.5 ± 0.7) × | (2.3 ± 0.71) × | (9.8 ± 1.2) × |
| (H4N2) | 10⁴ | 10² | 10⁶ | 10³ | 10⁶ | 10³ |
| Fold | 2 × 10² | | 7 × 10² | | 2 × 10² | |
| improvement | | | | | | |
| Turkey/E119D | >3.6 × 10 | (1.7 ± 0.8) × | n.d. | n.d. | n.d. | n.d. |
| (H4N2) | | 10³ | | | | |

| | | | |
|---|---|---|---|
| Fold | >2 × 10³ | n.d. | n.d. |
| improvement | | | |
| | 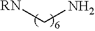 | 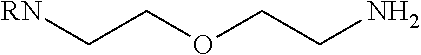 | 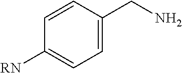 |

Note:
IC50 of Zanamivir for both Wuhan and Turkey/WT is 2 × 10⁴ nM.
IC50 of Zanamivir for WSN is 8 × 104 nM.
IC 50 of Zanamivir for Turkey/E119D is 2 × 10⁷ NM.

### Example 2

### Evaluation of Efficacy of Zanamivir-Polyglutamine Conjugates in Mice

Male Balb/C mice at 8 weeks (Jackson Laboratories, Bar Harbor, Me.) were used in this study. The mice were anesthetized with intraperitoneal avertin injection and dosed intranasally in one nostril with a 25 μL solution of either PBS (vehicle control), 1, 5, or 5a. Within 10 min, mice were then infected with 25 μL of virus solution in PBS (1,000 pfu/mouse) delivered in the same nostril. At 6, 24, and 48 h postinfection (p.i.), mice were again given PBS, 1, 5, or 5a.

Inhibitor doses were 0.028 μmol/kg for 1, an equimolar dose of 5a (0.028 μmol/kg on a 1 basis; 0.24 μmol/kg on a monomer basis), and 11 μmol/kg 5 (40-fold molar equivalency on a monomer basis). Group sizes were: PBS—6 mice, 5—3 mice, 5a—4 mice, and mock infection—3 mice. For WSN infection, 5 mice were given 1. For PR8-infection, 6 mice were given 1. Animals were euthanized with CO2 at 72 h post-infection (p.i). Whole mouse lung was harvested, rinsed in ice-cold homogenization buffer (50 mL of 1×PBS plus 150 μL of 35% BSA and 500 μL of a solution of 10,000 IU/ml penicillin G and 10,000 mg/mL streptomycin (JR Scientific, Woodland, Calif.)), flash-frozen on dry ice in 1 mL of ice-cold homogenization buffer, and stored at −80° C. until processing.

For PR8-infected mice, lungs were homogenized using a Branson Sonifier 250 with a 1/8″ tapered tip probe (Branson Ultrasonics, Danbury, Conn.). Total RNA was extracted from 170 μL of clarified lung homogenate using the PureLink Viral RNA/DNA Kit (Invitrogen, Carlsbad, Calif.) according to manufacturer's instructions. Viral RNA was eluted in Tris-EDTA buffer (pH 8.0) and stored at −80° C.

Quantification of viral RNA was performed using the RNA Ultrasense One-step qRTPCR System (Invitrogen) according to manufacturer's instructions after treatment with RNase-free DNase (Ambion, Austin, Tex.). Primer and probe to detect the encoding region for the M1 matrix protein were used at concentrations of 1,900 nM and 754 nM, respectively, in a total reaction volume of 40 μL.

Sequences of influenza A-specific primers and probe (IDT, Coralville, Iowa) were previously established A Roche LightCycler instrument was used for real-time reverse-transcriptase PCR using the following program: 45° C. for 30 min, 95° C. for 2 min, and 50 cycles of 95° C. for 5 sec, 55° C. for 10 sec, and 72° C. for 10 sec. All samples and a standard curve of serially diluted un-passaged virus were run on the same reaction plate. Levels of viral RNA in lung homogenates are expressed as threshold cycle (CT), determined using LightCycler® 480 System software v. 1.5.

To confirm that any residual polymer in homogenates did not interfere with RNA extraction, a sample of stock virus and a sample of homogenate from an untreated mouse were spiked with 5 before extraction and PCR. A standard curve of the spiked virus and CT value of the spiked homogenate were in agreement with that of the unpassaged virus. Thus, the observed CT values reflect robust purification.

For WSN-infected mice, lungs were homogenized using a Dounce homogenizer on ice. Viral titers of WSN in clarified murine lung homogenates were determined by 12-well format plaque assay and expressed in pfu/mL. To exclude the possibility that the reduced titers measured in treated groups were a result of residual 1 or 5a in lung homogenates, one uninfected mouse was dosed with each inhibitor. Lung homogenates from these mice were mixed in equal volume with that of infected but untreated (PBS control) mice. No significant difference in virus titer was observed; the reduction in titer seen with treated mice does indeed reflect in vivo inhibition.

Immune response studies were performed with 8-week old male Balb/C mice. Mice were split into two groups of four, anesthetized with intraperitoneal avertin and challenged with 40 μL of PBS or 40 μL of 1 mg/mL 5a at 0, 6, 24, and 48 h. After 4 weeks, mice were re-challenged with three administrations of 40 μL of PBS or 40 μL of 1 mg/mL 5a. Serum samples were collected “prechallenge” from a tail-vein 3 weeks before initial challenge. “Primary challenge” and “secondary challenge” samples were collected 10 days after initial challenge and secondary challenge. Serum samples were separated using BD Microtainer serum separator tubes (Becton Dickinson, Franklin Lakes, N.J.) and stored at −80° C.

ELISA to determine total and specific immunoglobulin levels in mouse serum was performed according to a modified literature procedure using Costar Universal Bind plates (Corning, Tewksbury, Mass.). Antibody pairs and standards (mouse IgG, IgM, and IgA) and TMB substrate were used directly from Ready-Set-Go Mouse Ig kits (eBioscience, San Diego, Calif.). For detection of 5a-, 5-, or 1-specific antibodies by ELISA, 50 μL of 0.01 mg/mL 5a or 5, and 50 μL of 0.1 mg/mL 2 were incubated overnight at 4° C. Capture antibodies were incubated according to manufacturer's instructions. For washing, 1% PBST (1% BSA (w/v) and 0.05% (v/v) Tween 20 in PBS) were used. Blocking (4 h, RT) and serum dilutions (100 μL total incubation volume) were performed with 2% PBST (2% BSA (w/v) and 0.05% (v/v) Tween 20 in PBS). After washing five times, HRP-conjugated antibody was incubated at RT for 3 h, and detection performed as per manufacturer's instruction. Serum from all experimental mice plus serum from an untreated but WSN-infected mouse (positive control collected at 2.5 weeks p.i.) were included on each plate. Sensitivity of the assay was 1.5 ng/mL for IgG, 0.7 ng/mL for IgM, and 0.7 ng/mL for IgA.

Results

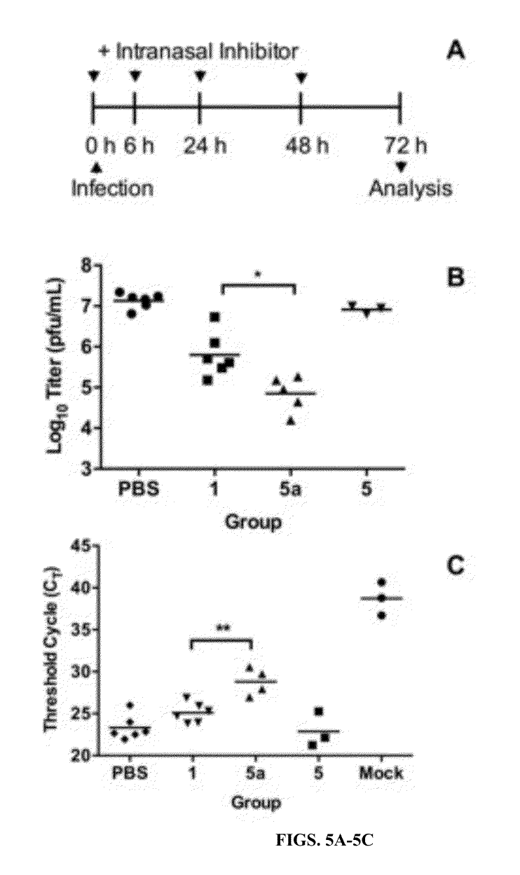

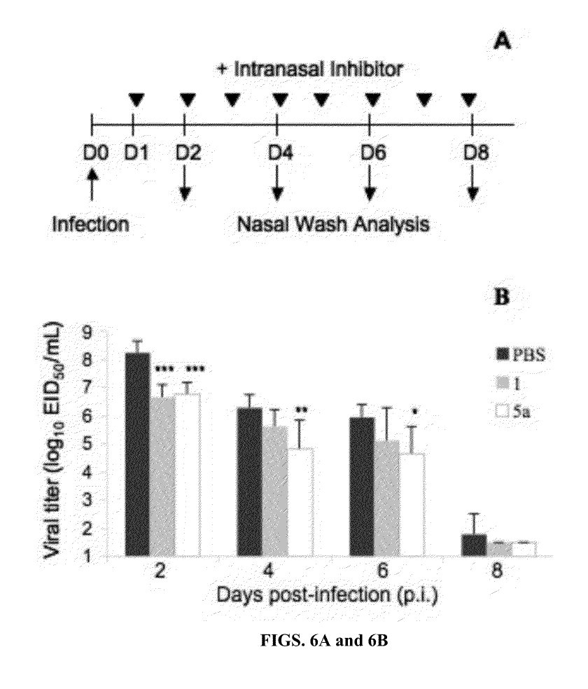

Mice were given doses of polymeric 5a, small-molecule 1, or PBS (as a control) intranasally, immediately followed by intranasal infection. At 6, 24, and 48 h post-infection (p.i.), the mice were again given 5a, 1, or PBS intranasally (FIG. 5A). Viral load was measured in lung homogenates at 72 h p.i.

Viral titers from lung homogenates of WSN-infected mice were determined using the plaque assay. Untreated mice had high titers of 107 pfu/mL (FIG. 5B). When treated with 1, the titers dropped 20-fold. Upon treatment with a molar equivalency (in terms of 1) of polymeric conjugate 5a, the titers plummeted 190-fold, whereas no decrease was detected from treatment with poly-L-glutamine (5) alone. Thus 5a is some 10-fold more potent than 1 at inhibiting WSN infection in mice.

For mice infected with PR8, lung homogenates was analyzed using qRT-PCR. Treatment with 5a reduced viral load 11-fold more than that of 1 (FIG. 5C), which correlates well with the above-referenced WSN study. Across both influenza strains and both analytical methods polymeric conjugate 5a was an order of magnitude more potent than small molecule 1.

In mice, intranasal delivery of fluids post-infection with influenza virus has been shown to exacerbate the disease. This fact can render experimental compounds less potent because they must inhibit significantly higher viral titers than presumed. Consequently, the data may even underestimate the potency of polymeric inhibitor 5a in the mouse model.

### Example 3

### Evaluation of Efficacy of Zanamivir-Polyglutamine Conjugates in Ferrets

Adult male Fitch ferrets, five months of age (Triple F Farms, Sayre, Pa.), serologically negative by hemagglutination-inhibition assay for currently circulating influenza viruses, were used in this study. Six ferrets per group were anesthetized with an intramuscular injection of a ketamine hydrochloride (24 mg/kg)-xylazine (2 mg/kg)-atropine (0.05 mg/kg) cocktail and infected intranasally with Nanchang virus at 105 EID50 in a final volume of 1 mL of PBS. Ferrets were sedated by Ketamine before intranasal delivery of 500 μL (250 μL per nostril) of 6 μmol/kg bodyweight of 5a in PBS; six control ferrets received vehicle (PBS) only. Ferrets receiving treatment with 1 were given 0.7 μmol/kg bodyweight in PBS administered intranasally. Ferrets received daily dosing of PBS, 5a, or 1 over a period of eight days beginning 24 h p.i. Ferrets were monitored daily for changes in body weight and temperature, as well as clinical signs of illness. Body temperatures were measured using an implantable subcutaneous temperature transponder (BioMedic Data Systems, Seaford, Del.). Virus shedding was measured in nasal washes collected on days 2, 4, 6, and 8 p.i. from anesthetized ferrets as previously described. Virus titers in nasal washes were determined in eggs and expressed as EID50/mL.

Results

The efficacy of 5a was evaluated in ferrets because this animal model of influenza infection is known to accurately reflect virus infectivity and antiviral activities in humans. On day 0, ferrets were infected with A/Nanchang/933/95 (Nanchang) virus, a clinically relevant human influenza strain. Beginning one day p.i., the ferrets were dosed daily with 5a, 1, or PBS (as a control). A nasal wash was collected from each ferret on days 2, 4, 6, and 8 to measure viral titer. On day 2 p.i., the ferrets given equimolar doses of 1 or 5a (in terms of 1) exhibited similar reductions in titer of 38- and 30-fold, respectively, compared to the PBS-treated group. Treatment with 5a continued to reduce viral titers significantly compared to PBS controls-30- and 20-fold on days 4 and 6 p.i., respectively, —whereas treatment with 1 did not. Thus, polymeric conjugate 5a is a more effective therapeutic agent than 1 in this highly relevant ferret model of influenza infection.

Finally, repeated dosing was examined to determine if 5a induces immune responses, which could render 5a ineffective. Although small molecules, such as 2, do not typically elicit an antibody response, as part of a polymeric conjugate they can behave as haptens and become immunogenic. To assess this possibility, mice were challenged intranasally for four days with a daily dose of PBS (as a control) or 40 μg of 5a (which was 40 fold higher than what was used to inhibit virus infection in mice). Ten days after the first administration serum samples were collected. To increase the probability of antibody induction, after four weeks the mice were re-challenged for three days with PBS or 40 μg of 5a daily, and sera were again collected ten days later. Using 2, 5, and 5a as capture antigens in an ELISA assay, we tested for the presence of specific IgG, IgM, and IgA in the serum samples. Only a background level of immunoglobulin was detected in the mice given 5a before, after primary, and after secondary challenge, the same as in control mice given PBS (Table 2).

TABLE 2
Drug-specific serum immunoglobulin ELISA titers
from mice treated with high-dose 5a or PBS
(as a control) were measured pre-challenge (“Pre-”),
10 days after primary challenge (“Primary”),
and 10 days after secondary challenge (“Secondary”).

| | Time point | Reciprocal specific antibody titers^{a} | | | | | |
|---|---|---|---|---|---|---|---|
| Capture | (relative to | IgG | | IgM | | IgA | |
| antigen | challenges) | PBS | 5a | PBS | 5a | PBS | 5a |
| 1 | Pre- | <20 | <20 | <20 | <20 | <20 | <20 |
| | Primary | <20 | <20 | <20 | <20 | <20 | <20 |
| | Secondary | <20 | <20 | 20 | 20 | <20 | <20 |
| 5 | Pre- | <20 | <20 | 20 | 20 | 20 | 20 |
| | Primary | <20 | <20 | 20 | 20 | 20 | 20 |
| | Secondary | <20 | <20 | 20 | 20 | 20 | 20 |
| 5a | Pre- | <20 | <20 | <20 | <20 | <20 | <20 |
| | Primary | <20 | <20 | 20 | <20 | 20 | <20 |
| | Secondary | <20 | <20 | 20 | 20 | 20 | 20 |
| 5b | Pre- | <20 | <20 | 40 | 40 | 20 | 20 |
| | Primary | <20 | <20 | 40 | 20 | 20 | 20 |
| | Secondary | <20 | <20 | 40 | 40 | 20 | 20 |

^{a}The titers are reported as the reciprocal of the least dilute sample with signal 2-fold above background and are the average of at least two measurements of each sample within each group (n = 3). Serum dilutions were 1:20, 1:40, and 1:100.

Even when 5d with 20 mole percent 2 was used as a capture antigen, only background levels of immunoglobulin were detected in 5a treated mice, as in PBS-treated mice. Thus, upon repeated challenge with a dose 40-fold higher than that used in the aforementioned infection studies, no neutralizing antibodies against 5a or any of its components were observed.

### Example 4

### PGN-ZA Binds to and Inhibits Viral NA

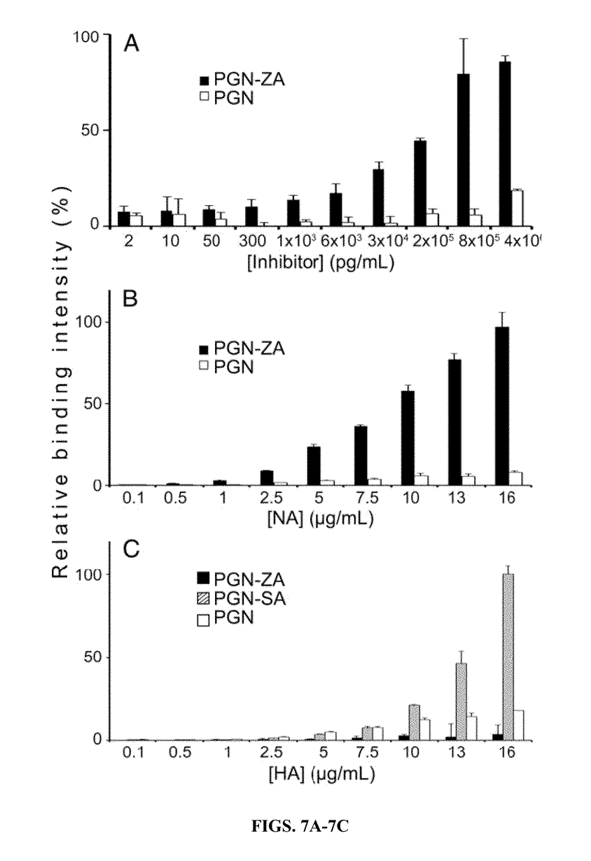

Influenza virus has two main surface glycoproteins, hemagglutinin (HA) and NA (21). Both of them bind to the terminal sialic acid of cell-surface. Since ZA is a sialic acid (SA) derivative and inhibits the enzymatic activity of NA, the effect of its conjugation to polyglutamine (PGN) via a flexible linker on its binding and inhibitory activities was evaluated. To characterize binding of PGN-ZA to whole virions, whole-virus ELISA binding assays were performed where PGN-ZA or PGN were immobilized to 96-well plates by UV cross-linking, incubated with influenza A/WSN33 (H1N1) (WSN), and then quantified using HRP-conjugated anti-H1 antibodies. PGN-ZA exhibited a concentration-dependent binding with saturation to the whole H1N1 viruses in the therapeutic range (FIG. 7A), whereas PGN itself showed no significant virus binding under the same conditions.

PGN-ZA's specific site of action was determined by measuring its binding to purified HA and NA proteins by means of ELISA. The polymer-attached drug displayed a dose-dependent binding to NA, but not to HA (FIGS. 7B and 7C). In contrast, multivalent polymeric SA conjugates (PGN-SA) exhibited specific binding to HA, as SA is the cognate ligand of HA (FIG. 7C). PGN by itself did not bind to either HA or NA. PGN-ZA was 3- and 10-fold more potent than ZA modified with the linker (ZA-linker) (ZA-linker's antiviral activity is similar to that of ZA itself (20)) in inhibiting NA activity of WSN and influenza A/PR/8/34 (PR8) viruses, respectively (Table 1). These data indicate that bare PGN has no appreciable interaction with HA, NA, or whole virions and that PGN-ZA specifically binds to NA and inhibits its enzymatic activity.

### Example 5

### PGN-ZA Inhibits Both Early and Late Steps of Influenza Virus Infection

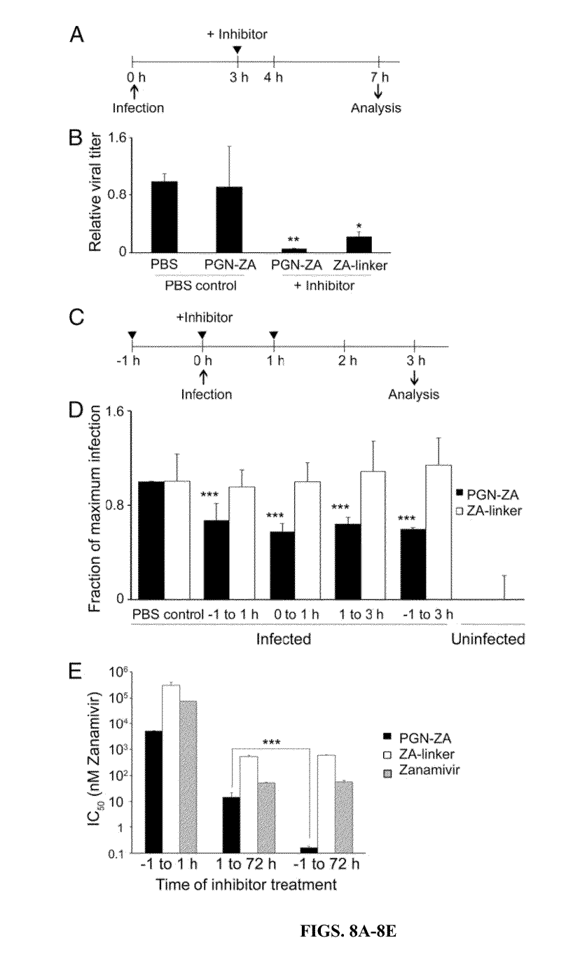

Since PGN-ZA inhibits NA, as does the monomeric ZA, it was expected that PGN-ZA would inhibit the release of newly synthesized virions. MDCK cells were infected at a multiplicity of infection (moi) of 2. Because newly synthesized viruses were released after about 4 h, PGN-ZA and ZA-linker were added 3 h post-infection (p.i.) to restrict inhibitory activity to the late phase of virus replication (FIG. 8A). At 7 h p.i., the culture supernatant was harvested, and the viral titer was measured by the plaque assay. Compared to the PBS control, addition of PGN-ZA and ZA-linker reduced the virus titer by some 90% and 80%, respectively (FIG. 8B). To control for the presence of leftover inhibitors in the collected supernatants (albeit at concentrations below IC₅₀ upon serial dilution), some PBS control samples were spiked with the same concentration of PGN-ZA just prior to the plaque assay. No significant reduction of virus titer was detected in those cases compared to the PBS control, confirming no interference from low concentrations of inhibitors remaining in the supernatants. These results show that PGN-ZA specifically inhibits the release of newly synthesized viruses from infected cells.

To test whether PGN-ZA inhibits early events of influenza virus infection, we performed time-of-addition experiments in a single-cycle infection (FIG. 8C). MDCK cells were infected with WSN virus at a moi of 20, and the inhibitors were added at different time points: −1 h, 0 h, or 1 h. The cell culture supernatants were harvested at 3 h p.i. before the completion of a single infection cycle. The cells were fixed, and expression of the viral proteins NP and M1 was quantified by flow cytometry. The fraction of infected cells decreased by 30-50% upon the addition of PGN-ZA (FIG. 8D). In contrast, for all the conditions tested, ZA-linker did not affect the fraction of cells infected. Thus PGN-ZA, unexpectedly, also specifically inhibits an early step of influenza virus infection.

To explore the relationship between PGN-ZA's inhibitory effects in the early and late steps of virus infection, a time-of-addition plaque assay was performed with the avian strain A/Turkey/MN/80 (TKY). The inhibitors were added in different time points of the assay: (i) early (−1 to 1 h p.i.), (ii) late (1 to 72 h p.i.), or (iii) both early and late (−1 to 72 h p.i.). When added during the late phase of plaque assay, PGN-ZA significantly reduced the number of plaques with an IC₅₀ of 14.8 nM (FIG. 8E). Remarkably, when the virus was exposed to PGN-ZA throughout the assay in both the early and late stages, the potency of PGN-ZA rose almost 100-fold to an IC₅₀ of 0.16 nM. The IC₅₀ values for the monomeric ZA and ZA-linker remained the same under both conditions, thereby revealing no additional benefit from introducing the monomeric inhibitors in the early phase of the infection. As expected, a reduction in the IC₅₀ values was also associated with a reduction in the sizes of the plaques (data not shown).

Taken together, the foregoing results indicate that (i) the multivalent PGN-ZA potently inhibits at least two distinct steps in influenza infection: an event early during the infection process, as well as the release of newly synthesized virions; (ii) monomeric ZA inhibits only virus release, and (iii) PGN-ZA's dual mechanism of action produces a synergistic inhibition of virus replication.

### Example 6

### PGN-ZA Inhibits Influenza Infection Through Neither Direct Virucidal Effect Nor Virus Aggregation

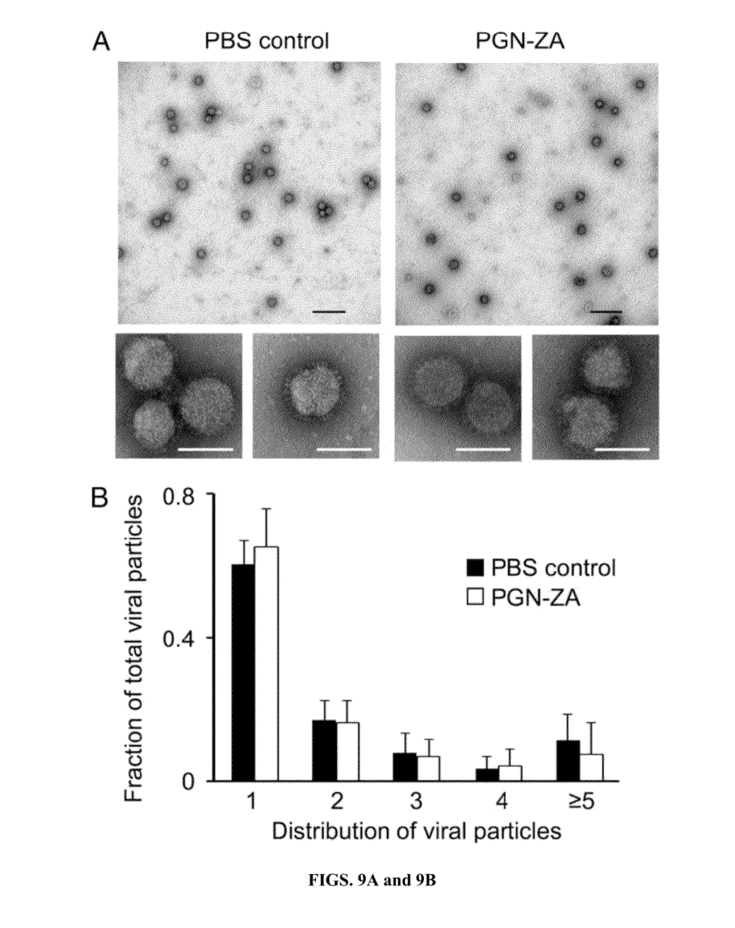

PGN-ZA may inhibit an early step of influenza virus infection through a direct virucidal effect and/or by aggregating viruses and thus preventing them from infecting target cells. To test these mechanisms, transmission electron microscopy (TEM) imaging was used to look for changes in viral envelope integrity and morphology upon PGN-ZA treatment. Purified WSN virus was filtered through a 0.2-μm filter and treated with either PGN-ZA or PBS for 1 h prior to staining with uranyl formate, followed by TEM imaging. As seen in high-magnification micrographs, PGN-ZA did not affect the morphology or envelope integrity of viral particles (FIG. 9A, lower panel). In addition, low-magnification micrographs (FIG. 9A, upper panel) were taken to determine the distribution of viral particles in clusters. From over 5,000 viral particles analyzed, no significant increase was observed in virus aggregation (clustering of two or more viruses together) upon PGN-ZA treatment (FIG. 9B), consistent with dynamic light scattering results. To rule out staining artifacts, phosphotungstic acid was also used to visualize the samples, and the data obtained corroborated those of the uranyl formate-stained samples (not shown). Thus, somewhat surprisingly, inhibition of the early step of influenza infection by PGN-ZA is not through a direct virucidal effect or aggregation of viral particles.

### Example 7

### PGN-ZA does not Affect Virus Attachment and Endocytosis

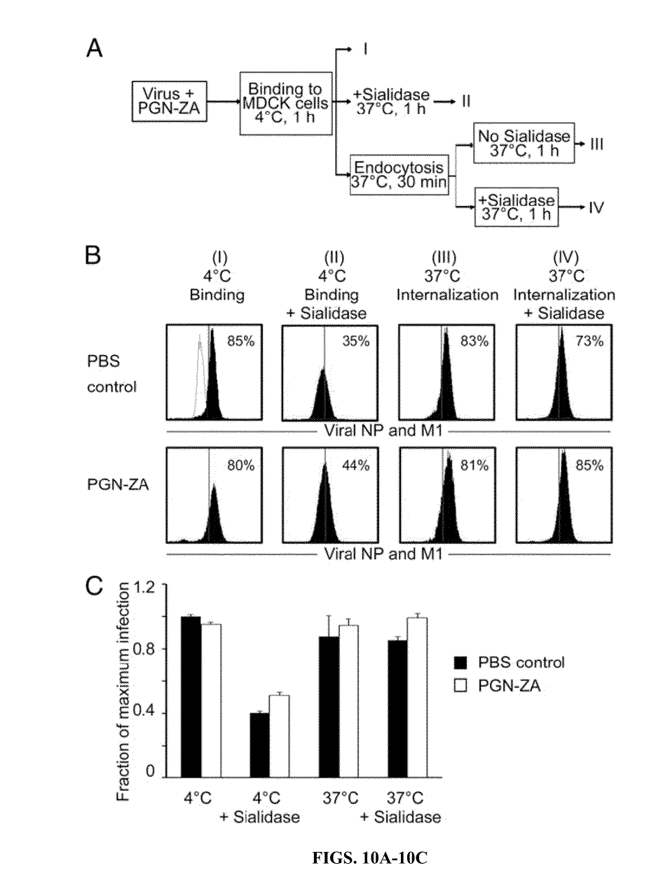

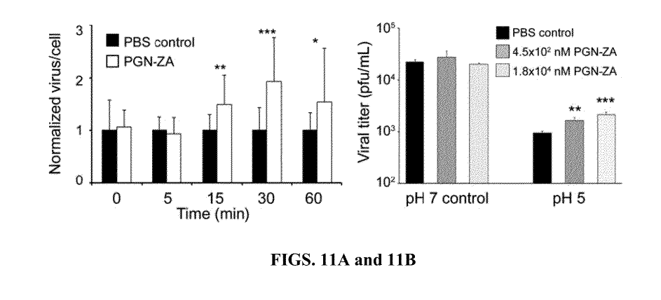

To examine whether PGN-ZA affects virus binding and endocytosis, flow-cytometry assay using labeled antibodies against viral NP and M1 (FIG. 10A). Virus attachment was measured by incubating WSN virus at moi of 20 with MDCK cells at 4° C., at which temperature endocytosis does not occur (FIG. 10A, Group I). To assay for endocytosis, the same cells were incubated at 37° C. for 30 min to allow the surface-bound virions to be endocytosed. Bacterial sialidase was later introduced into the system to remove surface-bound virions (FIG. 10A, Groups II and IV). Since internalized viruses are protected from sialidase activity, any cell-associated virus remaining after the sialidase treatment would presumably be that which has been internalized (FIG. 10A, Group IV). As shown in the left panel of FIG. 10B, PGN-ZA did not inhibit virus binding to MDCK cells. Expectedly, there was a significant drop in cell-associated viruses following sialidase treatment (FIG. 10B, Group II). PGN-ZA also did not affect virus endocytosis, as evidenced by the similar levels of cell-associated viruses with or without sialidase treatment of 37° C.-incubated cells (FIG. 10A, Groups III and IV). Statistical analysis of all four sets of conditions confirmed that the presence of PGN-ZA does not affect virus attachment and internalization (FIG. 10C). Consistently, hemagglutination inhibition assays also revealed that PGN-ZA did not affect virus binding to red blood cells. These results indicate that PGN-ZA does not inhibit binding of influenza viruses to the target cells or endocytosis of influenza viruses into the target cells.

### Example 8

### PGN-ZA Interferes with Intracellular Trafficking of the Endocytosed Viruses

To investigate PGN-ZA's effect on early steps of influenza virus infection, individual viral particles in MDCK cells fixed at different time points p.i. were imaged using fluorescence microscopy. The WSN virus was labeled with amine-reactive Alexa Fluor 647 dye; the virus retained infectivity and binding to red blood cells (data not shown). To synchronize infection, the viruses were first incubated with MDCK cells on ice for 60 min in the absence or presence of PGN-ZA. The mixture was then rapidly warmed to 37° C. to initiate infection. The MDCK cells were then fixed at 0, 5, 15, 30 or 60 min p.i. and stained with E-cadherin, Lysotracker and DAPI to visualize the cell boundary, the acidic compartments and nuclei, respectively. No apparent difference in the abundance of labeled viral particles was observed between the samples with or without PGN-ZA at t=0 min and t=5 min, concordant with the results of the flow cytometry-based binding experiments (FIG. 11A). However, from t=15 min onwards, a significant accumulation of viral particles was observed inside the cells with the PGN-ZA-treated samples, as compared to the PBS control (FIG. 11A). Notably, in PGN-ZA treated samples most of the viral particles did not co-localize with acidic compartments at t=15 and 30 min; and by t=60 min the accumulation of viral particles in the perinuclear region was clearly evident. Similarly, an accumulation of viral particles inside the cells at t=15 min was observed in the presence of amantadine, a known inhibitor of influenza virus acidification and fusion.

When an influenza virus is exposed to an acidic environment, HA is induced to undergo a conformational change. In the presence of a membrane, fusion occurs; in the absence of a membrane, the HA is irreversibly inactivated abolishing the viral infectivity. To investigate the ability of PGN-ZA to inhibit this process, the TKY virus was incubated at pH 5 in the presence or absence of PGN-ZA at 37° C. for 15 min. The level of infectious virus remaining after this acidic treatment was determined by serial titrations using the plaque assay. PGN-ZA blocked the pH 5-induced inactivation of virions 2-3 fold compared to the PBS control (FIG. 11B). In contrast, the viral titer did not change following a pH 7 incubation. Together, these results suggest that PGN-ZA inhibits the early steps of influenza virus infection by interfering with the intracelluar trafficking of the viruses once they are endocytosed.

### Example 9

### Effect of Polymer-Drug Conjugates on Onset of Drug Resistance

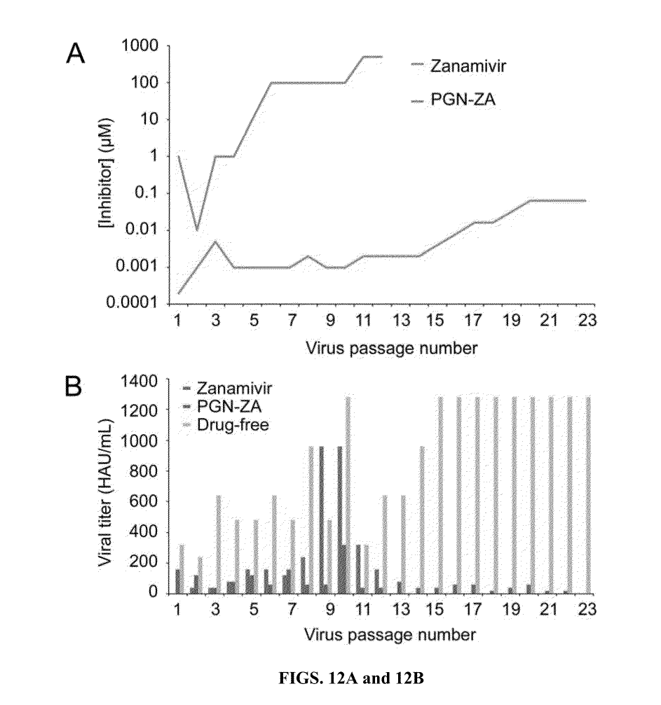

To investigate the effects of PGN-ZA on the appearance of drug resistance, A/Turkey/MN/80/833 (H4N2) virus (TKY) was passaged in MDCK cells in the presence of increasing concentrations of either zanamivir or PGN-ZA, and assayed the ability of the viruses to grow (FIG. 12A). The viruses were first diluted to MOIs of 0.001-0.1, and pre-incubated with either inhibitor for 1 h at RT. MDCK cells were inoculated with these virus mixtures for 45 min at 37° C., and the cells were then washed with pre-warmed PBS to remove any unbound or weakly bound viruses. The virus was grown for three days in medium containing the appropriate concentrations of ZA or PGN-ZA. Viruses were also grown under drug-free conditions in parallel as a control for mutations arising from adaptation to tissue culture. Viral growth for the three different conditions was titered on day three of each passage by a hemagglutination assay (FIG. 12B). Virus from the lowest MOI showing hemagglutinating activity was used for the next passage. The starting concentration for PGN-ZA was determined based on its IC₅₀ value by plaque reduction assay, and that of ZA was determined based on previous reports. The inhibitor concentration was increased in the subsequent passage if the virus appeared to have adapted to growing in the presence of the inhibitor. It was found that influenza virus adapted to growing in high concentrations (>100 μM) of monomeric ZA by passage 8 (FIG. 12B), whereas the growth of viruses under PGN-ZA selection pressure remained suppressed by low concentrations of PGN-ZA (<0.1 μM) (FIG. 12B) even after 23 passages. These results clearly demonstrate that PGN-ZA suppresses viral growth under drug selection pressure, and most likely delays the emergence of drug-resistant influenza viruses.

Hemagglutinin (HA) and NA on the influenza virus both bind to sialic acid on cell surface glycans. HA binds to sialic acid to initiate infection, whereas NA cleaves sialic acid to release newly generated viruses from cells. A functional balance exists between these two opposing activities for efficient virus replication, and adaptation to NA inhibitor selection pressure can be achieved by compensatory mutations in either HA and/or NA. Thus, to determine the effect of PGN-ZA on the emergence of drug resistance, the entire hemagglutinin (HA) and NA genes were sequenced from day 3 viral supernatants of that grown under ZA, PGN-ZA, or drug-free conditions. The results shown in Table 4 afford several findings on the timeline and mechanism of drug resistance progression.

TABLE 4

| | | | Ki (nM, based on ZA) | | IC₅₀ (nM, based on ZA) | |
|---|---|---|---|---|---|---|
| Virus isolates | HA1 | NA | ZA | PGN-ZA | ZA | PGN-ZA |
| Wild-type | — | — | 5.3 ± 0.4 | 1.1 ± 0.1 | 56 ± 8 | 0.16 ± 0.02 |
| Drug-free p23 | — | — | 8.8 ± 5.6 | 2.1 ± 0.9 | 38 ± 31 | 1.9 ± 1.8 |
| ZA p12 | | E119G | 340 ± 54 | 5.3 ± 0.1 | >>1.5 × 10⁵ | (1.5 ± 0.2) × 10⁴ |
| PGN-ZA p23 | R220G | G111D | 85 ± 36 | 5.6 ± 0.4 | >>1.5 × 10⁵ | (5.7 ± 5.9) × 10³ |
| | D241G | | | | | |
| Clone 167 | R220G | — | 0.84 ± 0.11 | 0.49 ± 0.05 | 130 ± 15 | 5.4 ± 4.3 |
| Clone 160 | R220G | — | 0.53 ± 0.08 | 0.57 ± 0.07 | 91 ± 13 | 15 ± 6.7 |
| Clone 123 | — | G111D | 1.1 ± 0.3 | 1.1 ± 0.3 | 65 ± 26 | 7.2 ± 3.9 |
| Clone 130 | — | G111D | 1.1 ± 0.07 | 0.62 ± 0.02 | 57 ± 30 | 3.5 ± 3.8 |
| Clone 126 | R220G | G111D | 0.92 ± 0.17 | 1.4 ± 0.14 | 350 ± 40 | 180 ± 150 |

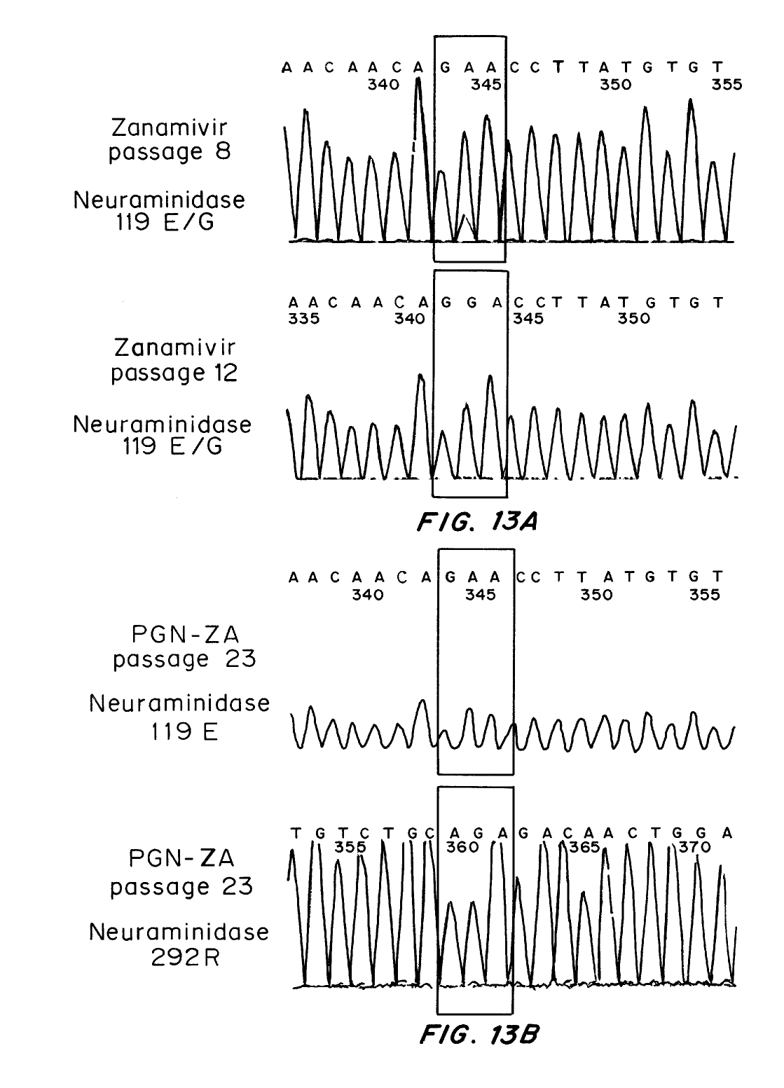

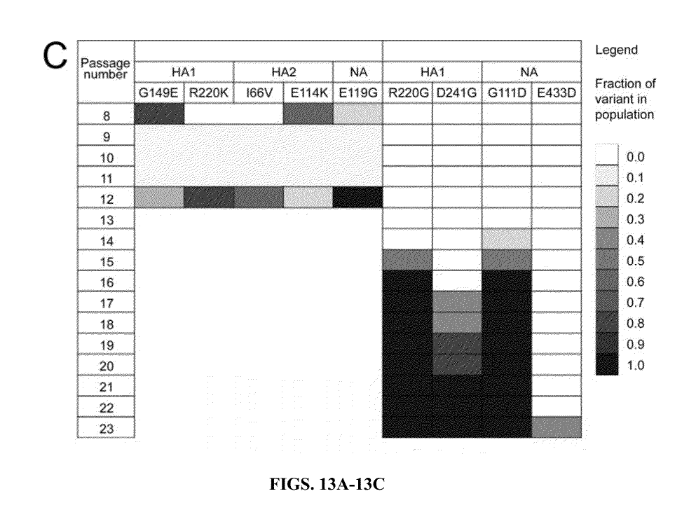

Passaging the virus for 23 rounds in the absence of inhibitors (hereforth termed DF23) resulted in the appearance of a mutation at residue 43 of the HA2 subunit (Ala to Val) (Table 4). Secondly, analysis of the NA gene confirmed the emergence of drug resistance after 8 passages in monomeric ZA (FIG. 13A). A mutation was found in residue 119, and this E119G variant (hereforth termed Z12) comprised 100% of the viral population by passage 12 (FIG. 13A). This is consistent with previous ZA selection studies using a variety of influenza subtypes, where mutation in glutamine 119 is well-documented to confer high levels of resistance to ZA. Additional mutations were also found in HA1 (R220K, G149E) and HA2 (166V, G114K), although these mutations did not reach full saturation in the population by passage 12 (FIG. 13C). Thirdly, for the viruses grown in PGN-ZA, the HA and NA sequence of viral supernatant from passages 8, and 12 to 23 was examined. Sequencing analysis revealed that the viruses remained free of changes in residue E119, and other known resistance-associated residues for all 23 passages (FIG. 13B). Instead, novel amino acid substitutions in HA1 (R220G and D241G), and in NA (G111D) appeared subsequently during passage 15-17 (FIG. 13C). All three mutations reached 100% saturation by the final passage (hereforth termed P23). From the P23 viral supernatant, 20 clones were isolated and each clone was cultured in the absence of inhibitors to test for stability of the genotype. The sequences of the clones are consistent with that of the original P23 supernatant. All the variants selected did not show any obvious defects in viral growth, or changes in receptor binding specificity.

The sensitivity of the viral variants Z12 and P23 to both ZA and PGN-ZA were assessed using plaque reduction assays (Table 4). The experiments yielded several interesting observations. Similar to the parental wild type virus (WT), the MDCK-passaged DF23 variant remained sensitive to both inhibitors (FIG. 51 and Table 4, row 2). As expected, Z12, the virus with the E119G substitution selected by 12 passages in ZA, was highly resistant to ZA (Table 4, row 3). Glutamine 119 is conserved among all influenza viruses and located in the enzyme active site of NA (21). The mutation from Glu to Gly causes the loss of a stabilizing ionic interaction between the guanidino moiety on ZA and the carboxylate of residue 119. Indeed, even at 150 μM of ZA, neither inhibition of plaque size nor plaque quantity was observed, which is at least 3000 times less sensitive than WT. Importantly, multivalent drug conjugate PGN-ZA was still somewhat effective against Z12; a reduction in plaque number was observed with IC₅₀ of around 15 μM. Next, we examined the inhibition of plaques of P23, virus selected by 23 passages in PGN-ZA (Table 4, row 4). Interestingly, P23 was resistant against monomeric ZA; like Z12, it was also at least 3000-fold less sensitive compared to WT. Although P23 was less sensitive against the multivalent drug conjugate compared to WT and DF23, PGN-ZA was still able to inhibit P23 replication with an IC₅₀ of 6 μM. In summary, these plaque reduction data indicate that the drug-selected variant Z12 and P23 was highly resistant to monomeric ZA, but still retained moderate sensitivity to PGN-ZA.

Subsequently, both variants' reduction in sensitivity in the plaque reduction assay was investigated to determine if it was predominantly caused by the amino acid substitutions in NA. To determine if their plaque reduction assay phenotype was correlated with the binding affinity of the inhibitors to viral NA, kinetic NA inhibition assays were performed to measure the variants' inhibition constants The results presented in Table 4 reveal several important observations. First, as expected, the NA of drug-selected variants P23 and Z12 bind monomeric ZA more weakly compared to the parental WT and MDCK-passaged DF23. The G111D mutation lowers ZA binding about 10-fold, whereas the E119G variant NA has a 70-fold decrease in ZA binding. Taken together with the plaque reduction results, the binding affinities observed for ZA correlates with its increased IC₅₀ in the plaque reduction assay. Second, in all the strains tested, PGN-ZA is a markedly more potent NA enzyme inhibitor than its monomeric counterpart ZA. Third, surprisingly, the viral NAs of P23 and Z12 bind almost just as strongly to multivalent PGN-ZA as WT and DF23; with almost two orders of magnitude improvement over that of ZA. The polymeric presentation of PGN-ZA completely compensates for the weakened binding in the NA of drug-selected variants. However, this does not reflect the reduction in sensitivity to PGN-ZA observed from the plaque reduction assays (Table 4).

To determine how each of the amino acid changes in HA and NA contribute to P23's drug resistance phenotype this, clones from the passage 15 virus grown in the presence of PGN-ZA were isolated. Amongst these, viral clones with the single mutation in either HA1 (R220G) or NA (G111D) were identified, and those with both mutations. Clones #160 and #167 possess the amino acid change R220G in HA1; clones #123 and #130 had the G111D mutation in NA; and clone #126 showed both the amino acid substitutions. With these clones, the effect of these single and double mutations on drug resistance was tested using the plaque reduction assay. The viruses with either the R220G or G111D were still strongly inhibited by both monomeric ZA and the multivalent PGN-ZA, with IC₅₀s comparable to the drug-free control DF23 (Table 3). Compared to these viruses with single mutations, clone #126 with both mutations was about 16- to 50-fold less sensitive to PGN-ZA, and about 4- to 6-fold less sensitive to ZA. These data indicate the synergistic effect of the two mutations, in particular against the multivalent PGN-ZA.

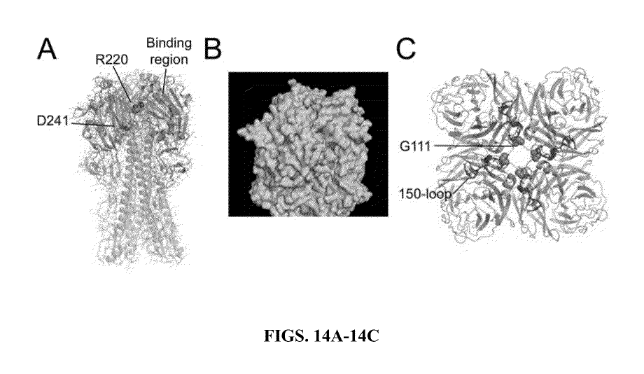

In order to better understand the molecular mechanism of resistance, the clones were also tested for the effect of ZA and PGN-ZA on NA enzymatic activity by using the NA inhibition assay. Surprisingly, we found that the NAs of the single and double mutants bind very well to both forms of ZA, with Kᵢ values in the nM range (Table 4). Structural modeling reveals the location of R220 and D241 to be facing the adjacent HA monomer unit (FIG. 14A-B), and calculations indicate that binding energy to both 2,3- and 2,6-sialic acid is not affected by both these mutations. As for G111D on NA, it is also located on the edge of the interface between NA monomer units (FIG. 14C). In the wild type NA, Gly 111 can come into contact with residue 141 of the neighboring unit and the 150-loop. Substitution with Asp will result in an atomic clash with residue 141, and may also affect the position of the 150-loop which can affect substrate binding to NA.

Taken together, results from the sequence analysis and phenotyping assays clearly indicate that PGN-ZA is able to delay the emergence of drug resistance by at least six passages, with a significantly better resistance profile than its monomeric predecessor ZA. Also, since both the HA1 and NA mutations in the PGN-ZA-selected virus emerged and reached saturation simultaneously, along with the observation that these two mutations acted synergistically in conferring resistance, the virus may need at least two mutations to occur for it to escape PGN-ZA inhibition. The probability of this event occurring is much lower than the single mutations required to gain resistance against the other existing antivirals, which can rationalize the delay in the emergence of drug resistance. Also, it is noted the ZA-resistant variants Z12 and P23 are still susceptible to low μM concentrations of PGN-ZA. In summary, our finding that the multivalent presentation of an existing small molecule drug ZA can minimize drug resistance opens up further possibilities in influenza antiviral drug design, and presents a potential therapeutic approach to counter the emergence of drug resistance.

We claim:

1. A conjugate comprising one or more neuraminidase inhibitors covalently coupled to one or more water-soluble, biodegradable polymers, wherein the one or more neuraminidase inhibitors are bound to the polymer via a carbamate linkage, the conjugate exhibits at least a 10 fold greater inhibition of sialidase than free neuraminidase inhibitor, the conjugate does not does not inhibit colloid interaction as determined by the hemagglutination assay, or combinations thereof.

2. The composition of claim 1, wherein the one or more neuraminidase inhibitors are selected from the group consisting of zanamivir, oseltamivir, laninamivir, peramivir, and combinations thereof.

3. The composition of claim 1, wherein the water-soluble biodegradable polymer is selected from the group consisting of polyglutamine, polyaspartate, homopolypeptides having an overall neutral charge under physiological conditions, and combinations thereof.

4. The composition of claim 1, wherein the molecular weight of the polymer is from 1,000 to 1,000,000 Daltons, preferably 10,000 to 1,000,000 Daltons.

5. The composition of claim 4, wherein the molecular weight is from 50-100 kDa.

6. The composition of claim 1, wherein the concentration of the one or more neuraminidase inhibitors is from about 5% to about 25% by weight of the polymer.

7. The composition of claim 6, wherein the concentration is 5%, 8%, 10%, 15%, 18%, or 20%.

8. The composition of claim 1, wherein the conjugate exhibits at least a 100 fold greater inhibition of neuraminidase than free drug.

9. The composition of claim 1, wherein the conjugate exhibits at least a 1000 fold greater inhibition of neuraminidase than free drug.

10. The conjugate of claim 1, wherein the neuraminidase inhibitor is covalently bound to the polymer via a carbamate linkage.

11. The conjugate of claim 10, wherein the neuraminidase inhibitor is bound to the polymer via a 5 carbon or 6 carbon linker.

12. A method of making the conjugate of claim 1, the method comprising coupling one or more neuraminidase inhibitors or derivatives thereof to a water-soluble biodegradable polymer.

13. A method of treating or preventing a viral infection, the method comprising administering to a patient in need thereof an effective amount of the conjugate of claim 1.

14. The method of claim 13, wherein the viral infection is selected from the group consisting of wild-type human or avian influenza, mutant human or avian influenza, respiratory syncythial virus, and combinations thereof.

15. A pharmaceutical composition comprising an effective amount of the conjugate of claim 1 and one or more pharmaceutically acceptable carriers.

16. The composition of claim 15, wherein the carrier is suitable for enteral administration.

17. The composition of claim 15, wherein the carrier is suitable for parenteral administration.
